(19) United States
(12) Patent Application Publication
(10) Pub. No.: US 2005/0101523 A1
(43) Pub. Date: May 12, 2005

(54) Amino acids from fish and soy proteins improve insulin sensitivity

(75) Inventors: Charles Lavigne, Sillery (CA); Helene Jacques, Sillery (CA); Andre Marette, St-Nicholas (CA)

Correspondence Address: Patterson, Thuente, Skaar & Christensen, P.A., 4800 IDS Center, 80 South 8th Street, Minneapolis, MN, 55402-2100, US

(73) Assignee: Universite Laval

(21) Appl. No.: 10/929,913

(22) Filed: Aug. 30, 2004

Related U.S. Application Data
(62) Division of application No. 10/018,189, filed on Mar. 18, 2002, now abandoned.

(30) Foreign Application Priority Data
Jun. 11, 1999 (CA) 2,274,414

(51) Int. Cl. A61K 38/17

(52) U.S. Cl. 514/2

(57) ABSTRACT

The present invention describes the use of fish protein (namely cod protein) and soy protein to improve the peripheral insulin resistance in a human or non-human animal. Fish and soy protein were administered to rats submitted to high-sucrose or high-fat/sucrose diets, which are animal models for diabetes. It was found that fish and soy protein efficiently control glucose utilization, and that fish protein is particularly effective in muscle tissue. This effect is not observed when rats are given casein. Fish and soy protein are therefore promising for controlling insulin-resistance, diabetes and complications resulting therefrom, such as obesity.

### PRIORITY INFORMATION

This application is a divisional of U.S. patent application Ser. No. 10/018,189, filed Mar. 18, 2002, entitled AMINO ACIDS FROM FISH AND SOY PROTEINS IMPROVE INSULIN SENSITIVITY, which claims priority to PCT application PCT/CA00/00714 filed Jun. 12, 2000 which claims priority to Canadian patent application 2274414 filed Jun. 11, 1999. The entire contents of U.S. patent application Ser. No. 10/018,189, are incorporated herein by this reference.

### FIELD OF THE INVENTION

This invention relates to compositions comprising fish and/or soy proteins, or comprising their hydrolysis peptides or amino acids, used for preventing insulin resistance or restoring normal insulin function in insulin-resistant subjects. Such compositions should be beneficial in preventing or remedying Type 1 and Type 2 diabetes, as well as the obesity that often accompanies the latter affliction, in human and non-human animals. This invention is especially effective in treating Type 2 diabetes.

### BACKGROUND OF THE INVENTION

Insulin resistance is characterized by an abnormally low response of the target cells to insulin, inducing high plasma insulin levels [1] and hypertriglyceridemia [2]. Several studies have demonstrated that the macronutrient composition of the diet is an important determinant of insulin sensitivity. Although most studies have examined the role of high-fat [3-7], low-soluble fiber [8] or high-sucrose diets [9] in the development of an impaired insulin action, relatively few studies have focused on the impact of dietary proteins. Up to now, a high-protein intake (60% of energy) has been reported to impair glucose metabolism in peripheral and hepatic tissues [10], but little information is available concerning the effect of different dietary protein sources. In this respect, when compared with casein, soy protein has been shown to decrease serum insulin concentrations in fasted normoglycemic rats [11]. Moreover, postprandial studies in rats [12], and in humans [13], showed a reduced postprandial insulin response to a single test meal containing soy protein compared with casein. Iritani et al. [14] further reported that dietary soy protein, in a saturated high-fat diet, may help to improve insulin sensitivity by increasing insulin receptor mRNA levels in liver and adipose tissues. Interestingly, cod protein has also been shown to reduce fasting plasma glucose compared with casein in normoglycemic rats [15]. However, little is known on the effects of cod and soy proteins on glucose tolerance, and on the postprandial plasma glucose and insulin response to a meal in rats maintained on controlled diets for a long-term period.

The components of dietary proteins responsible for these glucoregulatory effects are presumably the amino acids contained in the protein. However, it is not clear which amino acids are involved. On one hand, cod and soy proteins contain higher arginine levels than casein [16]. At levels found in dietary proteins, arginine is associated with a decrease of plasma insulin and the insulin/glucagon ratio [17]. This suggests that high arginine content in cod and soy proteins may contribute to reduce the fasting insulin response. On the other hand, high quantities of the amino acid lysine, that is present at higher levels in casein than in soy protein [16], have been shown to increase plasma insulin concentrations [18]. In this regard, Vahouny et al (1985) [11] measured in rats the effects on circulating glucose and insulin levels of diets in which the lysine/arginine ratio of the soy diet (0.9) was adjusted to that of the casein diet (2.0) by addition of L-lysine, and that of the casein diet was adjusted to that of the soy protein diet by addition of L-arginine. Fasting serum insulin levels in soy protein-fed rats were significantly lower than in casein-fed rats. Addition of arginine to the casein diet resulted in serum insulin levels comparable to those in the rats given the soy protein diet while addition of lysine to the soy protein diet resulted in serum insulin levels comparable to those in the rats given the casein diet. These results reinforce the role of arginine and lysine present in dietary proteins on the regulation of insulin sensitivity. However the mechanisms by which these amino acids exert their action on insulin sensitivity are still not clear.

At the cellular level, Traxinger and Marshall (1989) [19] observed a marked desensitization of the glucose transport system in adipocytes treated in a defined buffer containing 25 ng/ml insulin, 20 mM glucose, plus 15 amino acids found in Dulbecco's modified Eagle's medium (DMEM). Of the 15 amino acids, one group of 8 amino acids (glutamine, glycine, threonine, lysine, methionine, phenylalanine, valine, leucine) was found to be fully effective in mediating loss of insulin sensitivity. Interestingly, L-glutamine was as effective as total amino acids in modulating loss of insulin sensitivity, becoming the primary amino acid modulating glucose-induced loss of insulin sensitivity in adipocytes [19]. In skeletal muscles, plasma levels of amino acids, particularly the branched-chain amino acids (leucine, isoleucine) and threonine, may influence carbohydrate metabolism by decreasing insulin-mediated glucose uptake [20]. The infusion of amino acids during an euglycemic-hyperinsulinemic clamp decreased both whole body glucose oxidation and nonoxidative glucose disposal and forearm glucose disposal in normal fasted volunteers [21]. Patti et al (1998) [22] recently reported that exposure of cultured hepatic and muscle cells to a balanced mixture of amino acids down-regulated early steps in insulin action critical for glucose transport and inhibition of gluconeogenesis. It is also interesting to note that very recent studies have shown that taurine supplementation improved insulin sensitivity in animals models of insulin resistance [23, 24]. Although the mechanism involved in the effect of these amino acids on insulin sensitivity remains unclear at present.

Therefore, there is an interest in investigating whether fish and soy proteins, as compared with casein, could improve glucose tolerance, postprandial plasma glucose and insulin responses, whole-body glucose insulin action and peripheral glucose uptakes in high-sucrose or high-fat fed rats, which are well established models of insulin resistance.

### SUMMARY OF THE INVENTION

Based on the previous studies cited above, our working hypothesis was that cod and soy proteins exert beneficial effects on glucose tolerance, on peripheral insulin sensitivity, and on postprandial plasma glucose and insulin responses in rats maintained on controlled diets for a long-term period. To test this hypothesis, rats were fed controlled diets containing either casein, cod, or soy protein for 28 days. Various parameters of glucose tolerance and insulin sensitivity were measured during 1) an intravenous glucose tolerance test (IVGTT), 2) a hyperinsulinemic-euglycemic clamp, and 3) a test meal. Physiological curve responses of plasma C-peptide, glucagon, and triglycerides were also determined after the test meal.

For 28 days, male Wistar rats were fed isoenergetic high-sucrose diets containing either casein, soy protein or cod protein. In the fasting state, the cod protein- and soy protein-fed rats had lower plasma glucose and insulin concentrations, compared to casein-fed animals. Plasma glucose response after intravenous glucose bolus was lower after 10 and 20 minutes in cod protein- and soy protein-fed rats than in casein-fed rats, resulting in lower incremental areas under glucose curves and in a higher rate of glucose disappearance (Rd) with cod protein than with casein. Cod protein induced a lower insulin response to the glucose load, particularly during the late-phase insulin secretion (10 to 50 minutes), suggesting an improved peripheral insulin sensitivity in comparison with casein. In the test meal experiment, after a 12-hour fast, each dietary group received 5 g of their usual purified diet during 30 minutes. In the postprandial state, plasma glucose responses were similar regardless of protein origin. Postprandial plasma insulin, C-peptide and triglyceride concentrations were lower in cod protein- and soy protein-fed rats than in casein-fed rats at several time points following the test meal. Higher postprandial plasma arginine concentrations as well as lower branched-chain or essential amino acids could be involved in the improvement of insulin sensitivity in cod and soy protein-fed rats. On the basis of the metabolic responses to the responses to the three common dietary proteins, it may be concluded that, in comparison to casein, soy protein and cod protein improved insulin sensitivity and reduced fasting and postprandial plasma insulin response in rats fed high-sucrose diets.

Based on the data obtained in high-sucrose fed rats, it was of further interest to test the hypothesis that dietary cod or soy proteins may reduce or prevent the development of insulin resistance in a rodent model of obesity and insulin resistance. Consequently, a study was devised to test the effects of dietary cod and soy proteins compared with casein on peripheral insulin sensitivity of rats made obese by feeding a high-fat/sucrose diet. The high-fat/sucrose fed rat is a well-established animal model of insulin resistance reproducing the common form of the abdominal (visceral) obese insulin-resistant syndrome seen in humans [25-29]. Rodents fed on this diet rapidly develop whole body and skeletal muscle insulin resistance [30-32]. The effects of dietary proteins on basal and insulin-stimulated glucose uptake in individual insulin-sensitive tissues was also investigated by measuring the in vivo uptake of 2-[³H]-deoxy-D-glucose in skeletal muscles, heart, and adipose tissues. The present study shows that the consumption of cod protein (but not casein or soy proteins) fully prevents the development of skeletal muscle insulin resistance in diet-induced obesity. Furthermore, study conducted in vitro with L6 myocytes showed that cells treated with the amino acid mixture corresponding to plasma amino acid concentrations of cod protein-fed rats were more insulin-responsive than those treated with mixture representing casein. Our data suggest that the beneficial effect of dietary cod protein on insulin sensitivity is explained, at least in part, by a direct action of cod protein-derived amino acids on the myocyte glucose transport system.

In one aspect, the present invention provides compositions comprising fish and/or soy proteins, or hydrolysis peptides or amino acids derived therefrom, for use in controlling obesity complications in human or non-human animals.

The present invention further provides compositions comprising fish and/or soy proteins, or hydrolysis peptides or amino acids derived therefrom, for use in the treatment of hyperglycemia (diabetes) in human or non-human animals.

The disease diabetes mellitus is characterized by metabolic defects in production and utilization of insulin which result in the failure to maintain appropriate blood sugar levels. The result of these defects is elevated blood glucose or hyperglycemia. Research on the treatment of diabetes has centered on attempts to normalize fasting and postprandial blood glucose levels. Treatments have included parenteral administration of exogenous insulin, oral administration of drugs and dietary therapies.

Two major forms of diabetes mellitus are now recognized. Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The combination of the present invention is useful for treating both Type I and Type II diabetes. The combination is especially effective for treating Type II diabetes.

The compositions of the present invention are useful for controlling insulin-resistance, diabetes and obesity complications. For these purposes, the compositions of the present invention may be administered orally, parenterally (including subcutaneous injections, intravenous, intramuscular injection or infusion techniques), by inhalation spray, or rectally, in dosage unit formulations containing conventional non-toxic pharmaceutically acceptable carriers, adjuvants and vehicles.

Thus, the present invention further provides a method for controlling diabetes and obesity complications. The treatment involves administering to a patient in need of such treatment a composition comprising a carrier and a therapeutically effective amount of each compound of the present invention.

These compositions may be in the form of orally-administrable suspensions or tablets; nasal sprays; sterile injectable preparations, for example, as sterile injectable aqueous or oleaginous suspensions or suppositories.

### DESCRIPTION OF THE PREFERRED EMBODIMENTS OF THE INVENTION

The object, advantages and other features of the present invention will become apparent upon reading of the following non-restrictive description of the preferred embodiments thereof, given by way of example only with reference to the accompanying drawings.

### BRIEF DESCRIPTION OF THE FIGURES

FIG. 1. (A) Changes in plasma glucose concentrations in the fasting state and after iv glucose bolus in rats fed either casein, cod protein, or soy protein diet for 28 days. (B) Glucose area responses to intravenous glucose tolerance tests (IVGTT) in arbitrary units. (C) Changes in plasma insulin concentrations in the fasting state and after iv glucose bolus in rats fed either casein, soy protein, or cod protein diet. (D) Total insulin area responses to IVGTT. Groups bearing different letters for a given time point are significantly different (P<0.05). Areas are significantly different (P<0.05) if they do not share a common letter. Values are means±SE.

FIG. 2. (A) Glucose disposal rate (GDR) to maintain euglycemia during steady-state (60-120 min) insulin infusion in fasting state. (B) Plasma 2-deoxy-D-[³H]glucose disappearance rate (Kp). Rats were fed either casein, cod, or soy protein diet during 28 days. Bars represent means±SE of data obtained from 3-4 rats/group. Groups without common letter differ at P<0.05.

FIG. 3. (A) Changes in plasma glucose concentrations in the fasting state and after the test meal in rats fed either casein, cod protein, or soy protein diet for 28 days. Meals consisted of 5 g of their current diet. (B) Glucose area responses to the test meal in arbitrary units. (C) Changes in plasma insulin concentrations in the fasting state and the test meal in rats fed either casein, soy protein, or cod protein diet. (D) Insulin area responses to the test meal in arbitrary units. Groups bearing different letters for a given time point are significantly different (P<0.05). Areas are significantly different (P<0.05) if they do not share a common letter. Values are means±SE.

FIG. 4. (A) Changes in plasma C-peptide concentrations in the fasting state and after the test meal in rats fed either casein, soy protein, or cod protein diet for 28 days. (B) Plasma C-peptide area responses to the test meal in arbitrary units. (C) Changes in plasma glucagon concentrations in the fasting state and after the test meal in rats fed either casein, soy protein, or cod protein diet. (D) Plasma glucagon area responses to the test meal in arbitrary units. Groups bearing different letters for a given time point are significantly different (P<0.05). Areas are significantly different (P<0.05) if they do not share a common letter. Values are means±SE.

FIG. 5. Changes in plasma insulin-to-glucagon ratios (A) and plasma triglyceride concentrations (B) in the fasting state and after the test meal in rats fed either casein, soy protein, or cod protein diet for 28 days. Groups bearing different letters for a given time point are significantly different (P<0.05). Values are means±SE.

FIG. 6. (A) Changes in plasma free amino acids 30 minutes after the test meal in rats fed either the casein, soy protein or cod protein diet for 28 days. (B) Changes in plasma free amino acids 120 minutes after the test meal in rats fed either the casein, soy protein or cod protein diet for 28 days. Groups bearing different letters are significantly different (P<0.05).

Values are means±SE.

FIG. 7. Glucose infusion □ rate□ (GIR 60-120) to maintain euglycemia during steady-state (60-120 min)□ insulin infusion in fasted rats. Rats were fed either casein, cod, or soy proteins with the high-fat/sucrose diets during 4 wks. As a reference, the GIR 60-120 value of chow-fed is indicated by the dotted line. Values are means±SE for 7 to 9 rats in each group. Groups bearing different letters are significantly different at P<0.05.

FIG. 8. In vivo basal and insulin-stimulated 2-deoxy-D-glucose uptake in (A) white tibialis, white gastrocnemius and quadricep muscles and (B) soleus, red tibialis, red gastrocnemius and EDL muscles during euglycemic clamps. The mean value for insulin-stimulated chow-fed rats is represented by a dotted line. Bars represent means±SE of data obtained from 7 to 9 rats. Insulin-stimulated values bearing different letters are significantly different at P<0.05. No significant differences were observed for basal glucose uptake rates among high-fat fed and chow-fed groups.

FIG. 9. In vivo basal and insulin-stimulated 2-deoxy-D-glucose uptake in (A) heart and interscapular brown adipose tissue (BAT) and (B) white epididymal (WEpi) and white retroperitoneal (WRetro) adipose tissues during euglycemic clamps. The mean value for insulin-stimulated chow-fed rats is represented by a dotted line. Bars represent means±SE of data obtained from 7 to 9 rats. Insulin-stimulated values bearing different letters are significantly different at P<0.05. No significant differences were observed for basal glucose uptake rates among high-fat/sucrose fed and chow-fed groups.

FIG. 10. Effects of dietary proteins on (A) adipose tissue weights, (B) adipose tissue TNF-α expression, and (C) skeletal muscle TNF-α expression in high-fat fed rats. TNF-α protein levels were measured in extracts of epididymal adipose tissue and mixed gastrocnemius muscle as described in Materials & Methods (EXAMPLE 2, below). The mean value for insulin-stimulated chow-fed rats is represented by a dotted line. Bars represent means±SE of data obtained from 7-9 rats. No significantly differences were observed among high-fat/sucrose fed dietary groups by ANOVA analysis.

FIG. 11. Effects of casein-, cod protein-, or soy protein-derived amino acid mixtures on insulin-stimulated glucose uptake in L6 myocytes. Muscle cells were incubated for one hour with amino acids before measurements of glucose uptake as described in the Materials and Methods section in EXAMPLE 2, below. Results are expressed as insulin minus basal glucose uptake values. Basal glucose uptake rate (28.9±0.8 pmol*mg⁻¹*min⁻¹) was not altered by the amino acid mixtures. Insert shows insulin sensitivity index (EC50) calculated from individual dose-response curves. Data are means±SE of 4-5 separated experiments performed in triplicate. Groups bearing different letters are significantly different at P<0.05.

### EXAMPLE 1

### The Effects of Feeding Various Dietarv Proteins on Insulin Sensitivity and Glucose Tolerance in Rats

### MATERIALS AND METHODS

Animals. Male Wistar rats (Charles River, St. Constant, QC, Canada) weighing 240 g on arrival were individually housed in wire-mesh cages in a temperature- and humidity-controlled room with a daily dephased 12:12-h light-dark cycle (lights on at 2200 to 1000). Upon arrival, all rats were fed a grounded nonpurified commercial diet (Purina rat chow; Ralston Purina, Lasalle, QC, Canada) for at least 6 days. At the end of this baseline period, rats were divided into three groups of the same average weights. Purified diets and tap water were provided ad libitum for 28 days. Food intake was estimated every day by subtracting the food spillage weight from the initial food weight, and body weight was measured weekly. The animal facilities met the guidelines of the Canadian Council on Animal Care, and the protocol was approved by the Animal Care Committee of Laval University.

Diets. After the baseline period, all animals were assigned to one of the three purified powdered diets varying in protein source, namely casein, cod protein, soy protein, and were fed for 28 days. The composition of each purified diet is detailed in Table 1. Those diets have been recognized to induce higher fasting plasma glucose and cholesterol in casein-fed rats compared with cod protein- and soy protein-fed rats [15]. All diet ingredients, except vitamin mix (Teklad, Madison, Wis.) and cod protein, were purchased from ICN (Cleveland, Ohio.). Cod protein was prepared in our laboratory by freeze-drying cod fillets, followed by a 24-h delipidation using diethylether as solvent in a Soxhlet-type apparatus (Canadawide Scientific, Montreal, QC, Canada). Ingredients for the purified diets were mixed and stored at −20° C. until used. The energy content of the diets was measured with an automatic adiabatic calorimeter (model 1241; Parr Instruments, Moline, Ill.). Diets were found to be isoenergetic in the casein (19.91 kJ/g), soy protein (19.95 kJ/g), and cod protein (19.66 kJ/g) diets.

The protein content (N*6.25) was determined with a Kjeldahl Foss autoanalyzer (model 1612; Foss, Hillerod, Denmark). The level of protein in the purified diets was adjusted to an isonitrogenous basis at the expense of carbohydrates.

Experimental protocols. At day 25, rats were cannulated via the jugular vein (IVGTT and test meal experiments) and the carotid artery (hyperinsulinemic-euglycemic clamp experiment) under isoflurane anesthesia. Food intake was near normal postoperatively, and all rats were within 4% of surgery weight on the day of the study. Blood samplings were carried out in a 15×30-cm open plastic box to which rats were accustomed and in which they remained undisturbed during the experiment. Experiments 1, 2, and 3 were evaluated in separate groups of animals.

Experiment 1: IVGTT. At day 28, after a 12-h fast, 10 rats/dietary group were injected with 1.5 ml/kg body wt of a 35% glucose solution dissolved in saline as a bolus via the jugular catheter. The catheter was then flushed with saline. Blood samples (300 μl) were drawn through the catheter with EDTA-containing syringes (1.5 g/l blood) before (0 min) and 2, 5, 10, 20, and 50 min after the glucose load and were stored on ice. The plasma was separated by centrifugation and was stored at ±80° C. until analysis. All erythrocytes were pooled, resuspended in saline, and injected in the animals after the 20- and 50-min samples.

Experiment 2:Hyperinsulinemic-eul cemic clamp and in vivo 2-deoxv-D-glucose uptake. Whole body insulin action was determined by the hyperinsulinemic-euglycemic clamp procedure, as described previously [33]. Briefly, rats were fasted overnight, transferred to a quiet isolated room, and weighed. Unrestrained conscious animals were allowed to rest for 40 min before the first blood sample (300 μl). A continuous intravenous infusion of purified human insulin (Humulin R; Eli Lilly, Indianapolis, Ind.) was then started at the rate of4.0 mu*kg⁻¹*min⁻¹ and was continued for 140 min with a syringe pump (Razel, Stamford, Conn.) to achieve plasma insulin concentrations in the physiological range. Dextrose solution (25%) was infused through the venous line at a variable rate to maintain blood glucose at the initial value. Blood samples (40 μl) were taken from the carotid artery catheter at 5-min intervals to monitor plasma glucose concentrations using an Elite glucometer (Bayer, Etobicoke, ON). Every 20 min, an additional 300 μl of blood was withdrawn for later determination of plasma insulin levels. Erythrocytes were suspended in saline and reinjected into the animals to prevent a fall in the hematocrit and minimize stress. Insulin action within in vivo individual muscle was determined as described previously [33]. Briefly, the nonmetabolizable glucose analog 1,2-[³H]2-deoxy-D-glucose (2-[³H]DG) and D-[¹⁴C]sucrose (NEN Du Pont, Boston, Mass.) were administered together in an intravenous bolus 20 min before the end of the clamp. Blood samples were drawn at 5, 7.5, 10, 12.5, 15, 17.5 and 20 min after bolus administration for determination of radiolabeled 2-[³H]DG and [¹⁴C]sucrose. The plasma concentrations of 2-[³H]DG after the single injection were plotted on a semilogarithmic scale, and the rate of 2-[³H]DG disappearance from plasma was calculated from the slope obtained by a linear regression analysis, as described previously [34, 35]. At the completion of the clamp, rats were rapidly killed by decapitation, and the red gastrocnemius muscle was rapidly removed, frozen in liquid nitrogen, and stored at −80° C. for subsequent analysis. Tissue samples (50-100 mg) were dissolved in 1 ml of Solvable (NEN) at 55° C. for 16 h. Thereafter, hydrogen peroxide (30% solution) was added to decrease quenching, followed by the addition of 8 ml of scintillation fluid (BCS; Amersham, Mississauga, ON, Canada). The accumulation of 2-[³H]DG in muscle, corrected for extracellular space with [¹⁴C]sucrose, was used as an index of glucose uptake rates, as described by others [34, 35].

Experiment 3: Test meal. The experimental diet and jugular cannulation protocols were similar to those described in the IVGTT protocol. At day 28, after a 12-h fast, and at the beginning of the dark period, 10 rats/dietary group received 5 g of their assigned experimental diet for 30 min. After that time, any uningested food was removed. Blood samples were obtained before the beginning of the test meal (−30 min) and at 0 (end of the meal), 30, 60, 120, and 240 min. Blood samples for C-peptide and glucagon determinations (300 μl) were collected in tubes containing 250 kallikrein inhibitor units (Trasylol; Miles, Etobicoke, ON, Canada) at −30, 30, and 120 min only because of limited amounts of blood volumes. The plasma was separated by centrifugation and stored at −80° C. until further analysis. All erythrocytes were pooled, resuspended in saline, and reinjected in the animals after the 30-and 120-min samples.

Analytical methods. Plasma glucose levels were analyzed using a glucose oxidase method (YSI 2700 Select; Yellow Springs Instruments, Yellow Springs, Ohio), and plasma insulin, C-peptide, and glucagon levels were measured with a RIA method (Linco Research, St. Charles, Mo.) using rat insulin, C-peptide, and glucagon standards. Triglycerides were assayed by an enzymatic method using a reagent kit from Boehringer Mannheim (Montreal, QC, Canada). Incremental areas under the curves obtained during IVGTT and test meals were calculated with a computer graphic program with 0- and −30-min time points as baseline values, respectively.

IVGTT insulin response areas were distinguished as the first phase (0-10 min) and second phase (10-50 min) postinjection. ³H and ¹⁴C activities in aliquots of plasma and of dissolved tissue samples were determined by a liquid scintillation counter (Wallach 1409) using a dual-label counting program. Amino acid concentrations of crude protein and plasma samples were determined as reported by Galibois et al [36] and were analyzed by ion-exchange chromatography using a Beckman amino acid analyzer (Palo Alto, Calif.) model 6300.

Statistical analyses. Data were analyzed with the general linear model program on the SAS statistical package for personal computers. Data obtained from serial sampling were analyzed using ANOVA with repeated measures, with time as the repeated variable. Physiological parameters, food intake, hepatic insulin extraction, and incremental areas under the curve were analyzed using ANOVA. Individual between-group comparisons were performed using Duncan's new multiple range test after the ANOVA. Differences were considered significant at P<0.05. All results are presented as means±SE.

### RESULTS

Table 2 shows the amino acid composition of the tested dietary proteins. Casein contained the highest amounts of proline, tyrosine, and valine, whereas cod protein contained more alanine and lysine. Notably, the arginine level of cod and soy protein was two times that of casein. The levels of glycine and aspartic acid were also higher in soy protein and cod protein than in casein. The sum of branched-chain amino acids (leucine, isoleucine, and valine) was higher in casein than in cod and soy proteins, whereas the sum of essential amino acids was higher in the animal proteins, casein and cod protein, than in soy protein. Moreover, the lysine-to-arginine ratio was higher in casein (2.4) than in soy protein (0.9) and cod protein (1.5). After 28 days of dietary treatment, rats displayed comparable daily food intake and body weight gain regardless of the protein source (Table 3). The food intake for the last meal (experiment 3, test meal) was similar between the protein groups. After 4 wk of feeding, fasting plasma glucose and insulin were lower in cod protein- and soy protein-fed rats than in casein-fed rats (10 and 50%, respectively, P<0.05; Table 3).

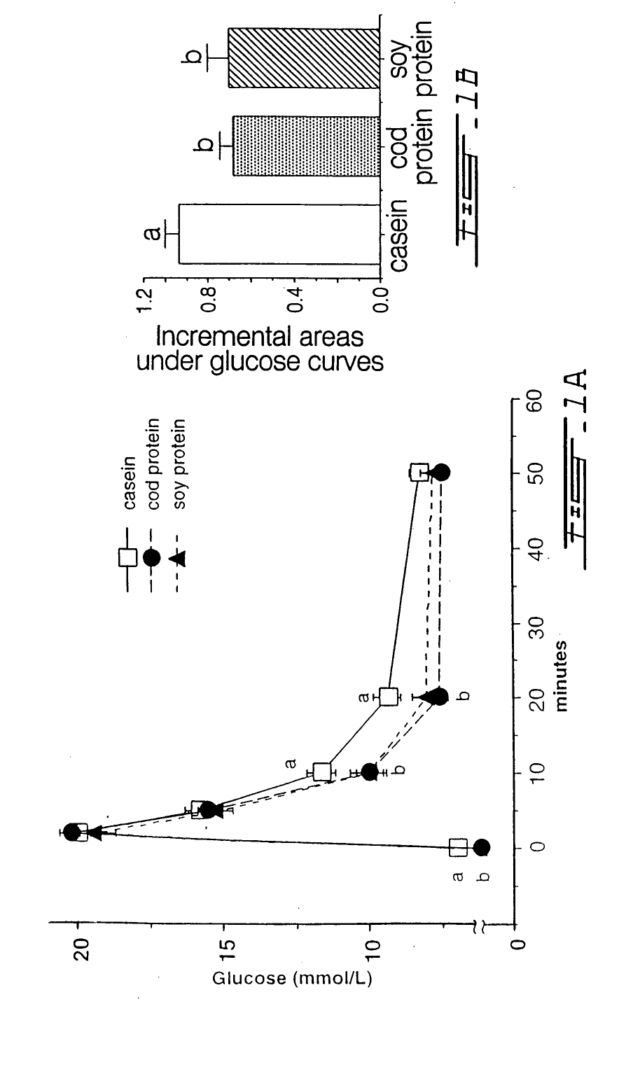

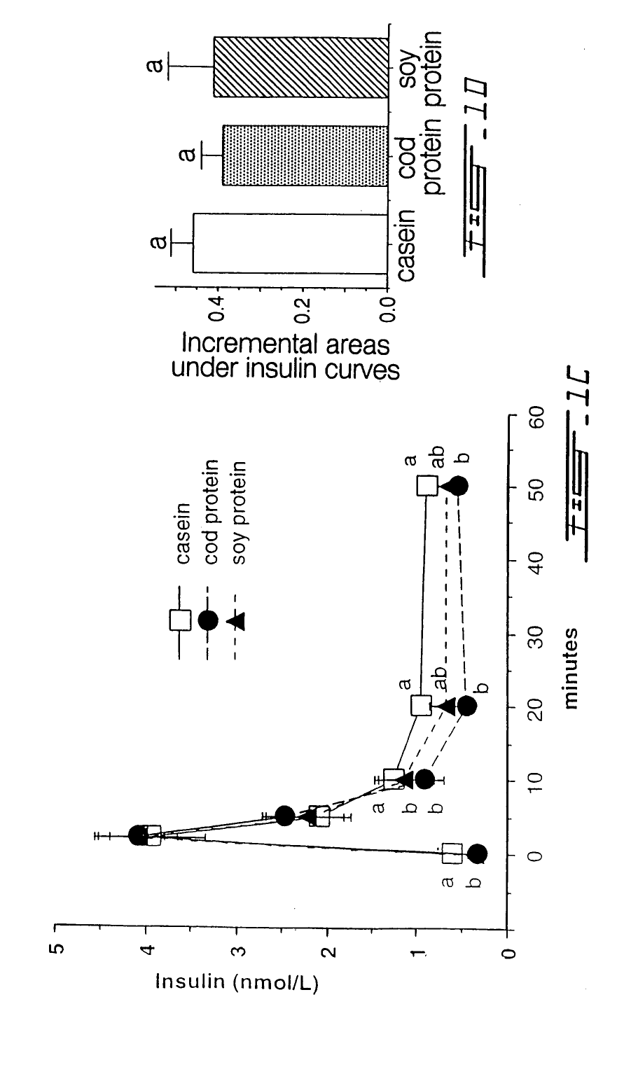

Plasma glucose and insulin responses and incremental areas under the glucose and insulin curves during IVGTT are shown in FIG. 1. Cod and soy protein diets resulted in significantly (P<0.05) lower plasma glucose 10 and 20 min after the intravenous glucose load compared with the casein diet (FIG. 1A). Cod (24%, P<0.05) and soy (22%, P<0.05) proteins induced smaller incremental areas under the glucose curves than casein (FIG. 1B). Rats fed cod protein, when compared with casein, displayed a lower insulin response (P<0.05) to the glucose load during the late-phase insulin secretion (10-50 min; FIG. 1C). Soy protein-fed rats displayed an intermediate response. However, the total incremental areas under the insulin curves (FIG. 1D) were similar between protein groups. We next divided the insulin response during IVGTT into an acute phase (first phase; 0-10 min) and a late phase (second phase; 10-50 min) to further dissect out β-cell function [37]. No significant differences were found between the experimental groups during the first phase (casein, 0.24±0.04 arbitrary units; cod protein, 0.29±0.03 arbitrary units; soy protein, 0.24±0.04 arbitrary units). However, the second-phase incremental areas under the insulin curves were significantly higher in the casein group compared with the cod protein group (0.22±0.04 vs. 0.07±0.02 arbitrary units, respectively, P<0.05), whereas soy protein-fed rats showed an intermediate response (0.15±0.05 arbitrary units).

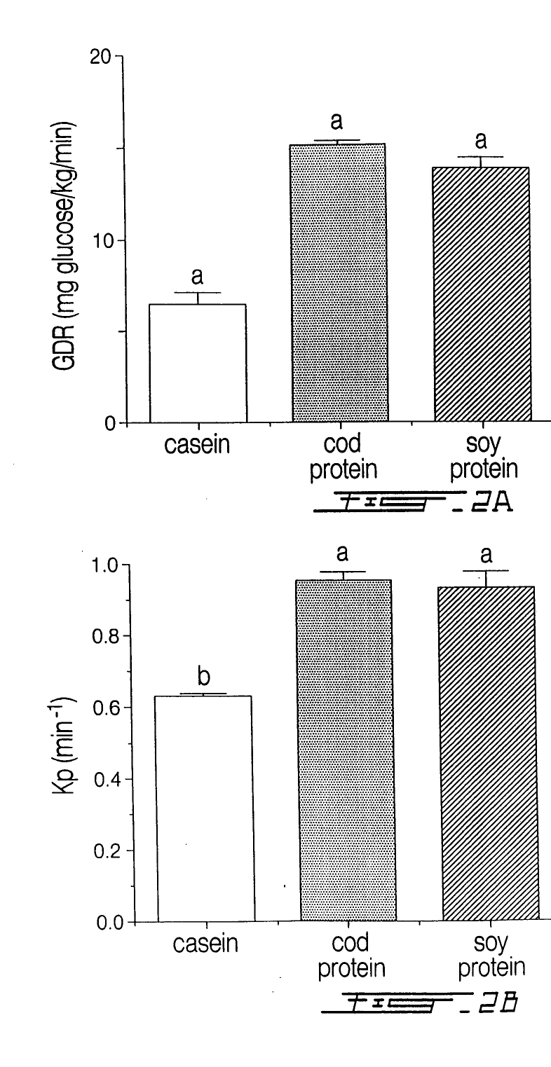

To see whether the improved glucose tolerance of cod protein- and soy protein-fed rats was associated with an increased peripheral insulin sensitivity, whole body and peripheral tissue glucose utilization was next measured during an hyperinsulinemic-euglycemic clamp. As depicted in FIG. 2A, both cod protein- and soy protein-fed rats displayed increased insulin sensitivity compared with rats fed casein. Plasma 2-[³H]DG disappearance rate was also higher in cod protein-fed (34%, P<0.05) and soy protein-fed (32%, P<0.05) rats than in casein-fed rats (FIG. 2B). In accordance with increased peripheral action of insulin in cod protein- and soy protein-fed rats, higher rates of insulin-stimulated glucose uptakes were observed in red gastrocnemius muscle of these rats (472±26 and 412±18 nmol*min⁻¹*g⁻¹, respectively) compared with casein-fed animals (213±24 nmol*min⁻¹*g⁻¹, p<0.05).

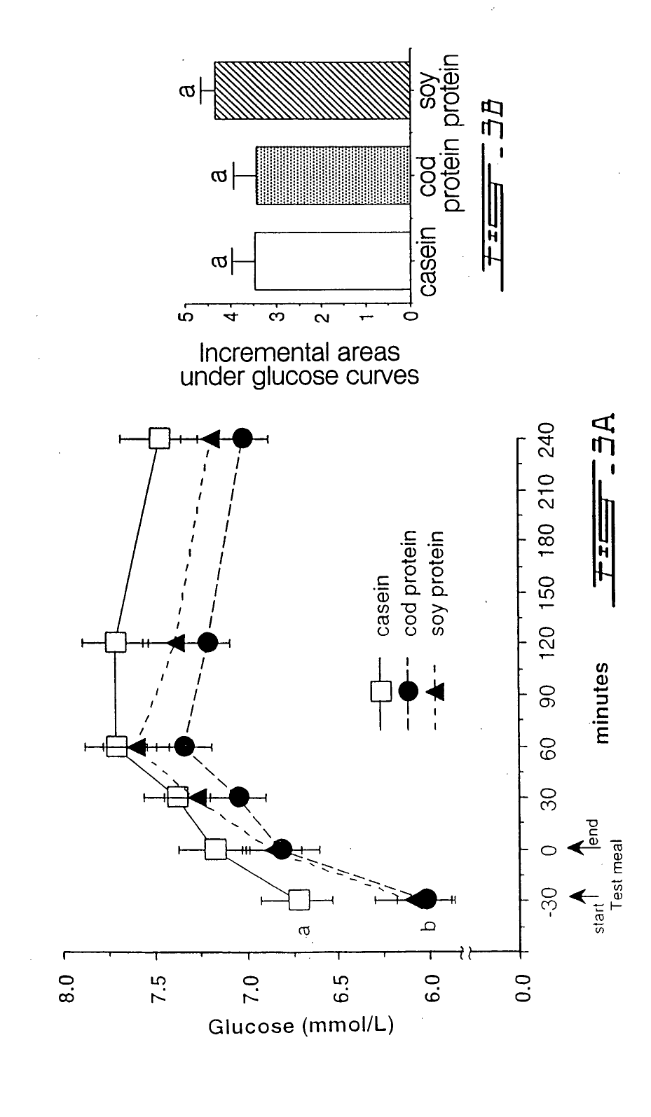

FIG. 3 shows fasting and postprandial plasma glucose and insulin responses and incremental areas under the glucose and insulin curves of plasma collected before and after the test meal in animals fed the diets for 4 wk. Postprandial plasma glucose reached a peak response after 1 h (FIG. 3A), and the incremental areas under the glucose curves after the test meal (FIG. 3B) were similar regardless of the protein consumed. Postprandial plasma insulin concentrations were lower (P<0.05) in soy protein-fed rats than in casein-fed rats at several time points (30, 60, and 120 min) after the test meal. Postprandial plasma insulin concentrations
were lower (P<0.05) in cod protein-fed rats compared with casein-fed rats immediately after the test meal (0 min) and 30 and 60 min after the test meal (FIG. 3C). The incremental areas under the insulin curves were significantly lower (P<0.05) with cod protein (25%) and soy protein (35%) than with casein (FIG. 3D).

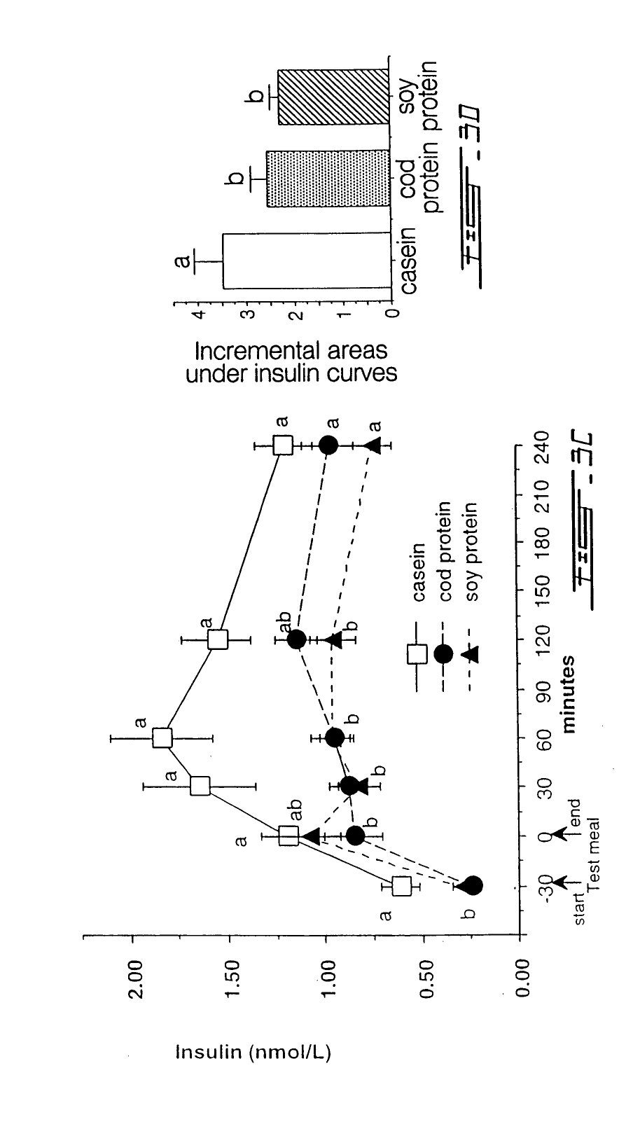

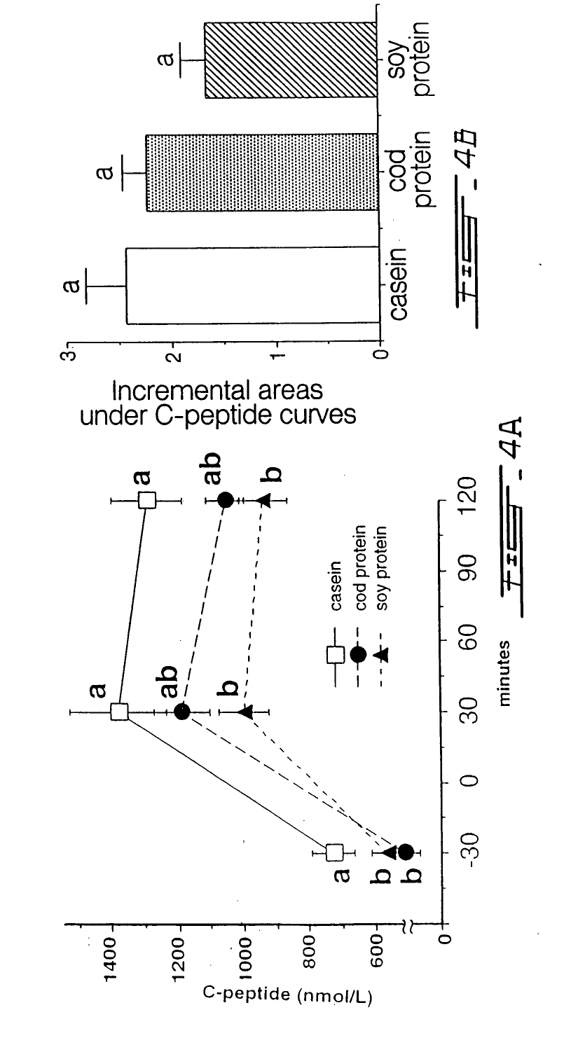

The plasma C-peptide curve responses are illustrated in FIG. 4A. In the fasting state (−30 min), C-peptide concentrations were lower in rats fed soy (23%, P<0.05) and cod proteins (30%, P<0.05) compared with rats fed casein. In the postprandial state, plasma C-peptide concentrations were lower (28%, P<0.05) at both 30 and 120 min after the test meal in soy protein- compared with casein-fed rats. Plasma C-peptide concentrations were intermediate at these two time points in cod protein-fed rats. The incremental areas under the C-peptide curves were similar whatever the dietary proteins consumed (FIG. 4B). Hepatic insulin extraction, as estimated by the molar C-peptide-to-insulin ratio, was significantly higher in the cod and soy protein groups in the fasting state and 30 min after the test meal than in the casein group (Table 4).

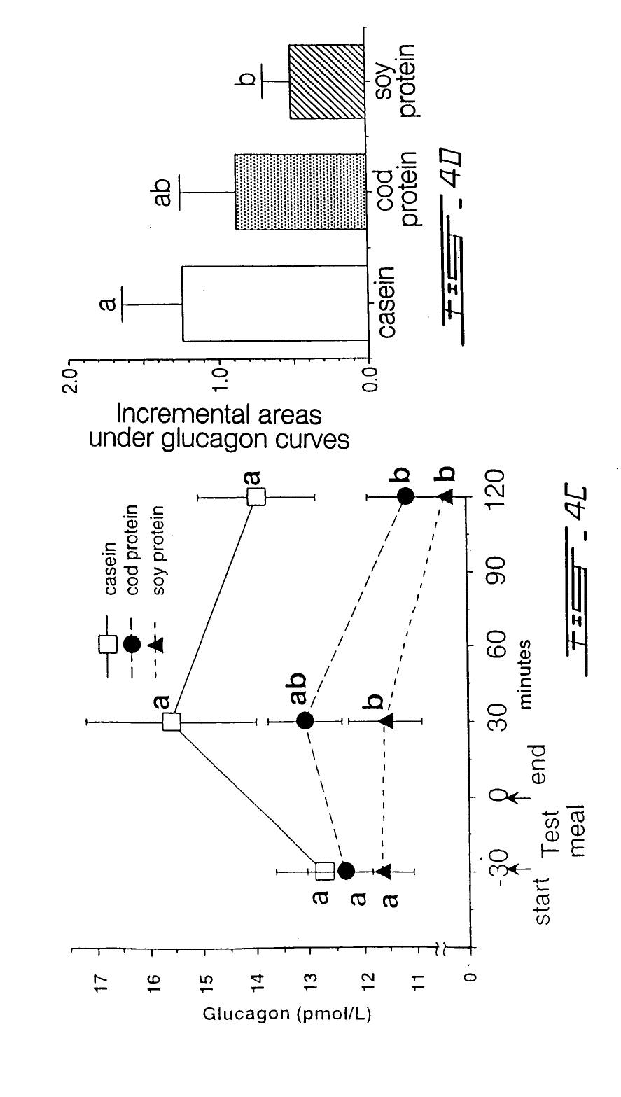

Plasma levels of the counterregulatory hormone glucagon are shown in FIG. 4C. Fasting glucagon concentrations were comparable among protein groups. After the ingestion of cod and soy protein meals, there was no significant increase in plasma glucagon concentration. In rats fed casein, the postprandial glucagon concentrations increased with a peak after 30 min, which was 26% (P<0.05) higher than that observed in soy protein-fed animals. Two hours after the test meal, the glucagon response was lower in cod protein- and soy protein-fed rats than in casein-fed rats by 20 and 25%, respectively (P<0.05). The incremental area under the glucagon curve was greater with casein than with soy protein (P<0.05; FIG. 4D). It is also noteworthy that the insulin-to-glucagon ratio was significantly (P<0.05) higher in rats fed casein than in those fed cod or soy proteins before and 30 min after the test meal (FIG. 5A).

Plasma triglyceride responses are shown in FIG. 5B. In the fasting state, cod protein- and soy protein-fed rats had lower (30 and 25%, respectively, P<0.05) plasma triglyceride concentrations compared with casein-fed rats. In the postprandial state, plasma triglyceride concentrations were lower 120 min after the test meal in cod protein- and soy protein-fed rats (25 and 40%, respectively, P<0.05) than in casein-fed rats, whereas 240 min after the test meal, plasma triglycerides were lower only in soy protein-fed rats compared with casein-fed rats (37%, P<0.05).

To gain insight the mechanism whereby glucose metabolism was affected by dietary protein, measurements of plasma amino acid levels were performed. Fasting plasma amino acid levels are presented in Table 5. In the fasting state, plasma L-aspartic acid and L-glycine concentrations of the soy protein-fed rats were higher than those of cod protein- and casein-fed rats. Plasma L-citrulline concentrations were higher with casein than with cod and soy proteins. Plasma L-histidine concentrations of cod protein-fed animals were lower than those of casein-fed and soy protein-fed animals. Plasma L-taurine concentrations were significantly higher in rats fed cod protein than in those fed casein, which were nevertheless higher than in those fed soy protein. However, L-arginine, L-lysine, lysine/arginine ratio as well as the plasma sum of total and total branched and essential amino acid concentrations were similar between dietary groups. Interestingly, plasma L-arginine was negatively correlated with fasting plasma C-peptide concentrations (r=−0.42, P=0.04, n=24) confirming the antisecretagogue role of plasma arginine in physiological concentration. No other fasted amino acid levels was correlated with measured parameters.

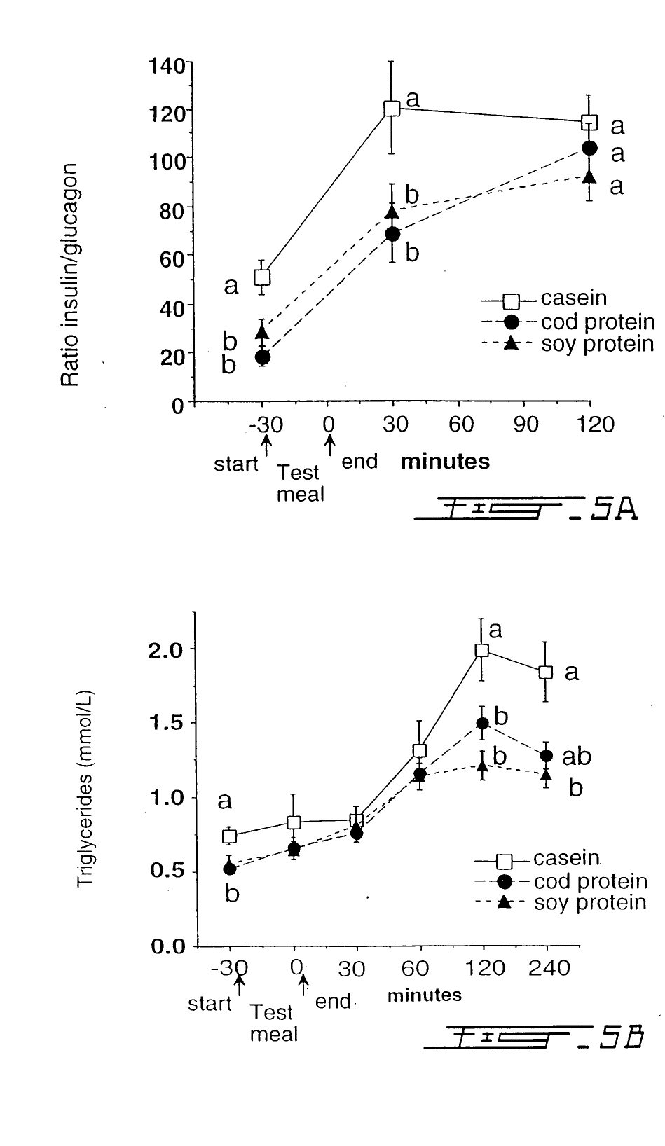

Only significant changes in plasma amino acid concentrations after the test meal (30 and 120 minutes vs. fasted −30 minutes) are illustrated in FIGS. 6A and 6B respectively. Changes in postprandial L-alanine (30 minutes), L-tyrosine (30 minutes), L-leucine (30 minutes), L-proline, and L-valine (30 and 120 minutes) were greater in rats fed casein than those in rats fed cod or soy protein. Changes in postprandial plasma L-arginine (30 and 120 minutes) resulted in lower concentrations in casein-fed rats compared with those in cod and soy protein-fed rats. Thus, changes in L-methionine (30 and 120 minutes) L-alanine and L-lysine (120 minutes) after the test meal were larger with casein- and cod protein-fed rats compared with those obtained in soy protein-fed rats. Postprandial plasma L-taurine changes (30 minutes) were higher in cod protein-fed rats whereas those in soy protein-fed rats, but casein-fed rats induced intermediate changes. After 120 minutes, L-taurine changes were significantly higher in cod protein group compared to casein and □ soy proteins groups. Thirty (30) and 120 minutes after the □ meal, sums of branched free amino acids were higher in casein fed group (30 minutes: 515±48 μmol/L, P<0.05; 120 minutes: 492±6□0 μmol/L, P<0.05) compared to cod (30 minutes: 375±□33 μmol/L; 120 minutes: 385±24 μmol/L) and soy protein (30 minutes: 393±□□25 μmol/L; 120 minutes: 393±□33 μmol/L). □□ Postprandial sums of essential free amino acids were higher in casein fed group (30 minutes: 1552±□122 μmol/L, P<0.05; 120 minutes: 1487±121 μmol/L, P<0.05) compared to cod (30 minutes: 1255±97□μmol/L; 120 minutes: 1155±□71 μmol/L) and soy protein (30 minutes: 1207±57 μmol/L; 120 minutes: 1160±71 μmol/L). Postprandial plasma L-alanine was correlated with posprandial plasma glucagon both after 30 (r=0.63, P=0.0005, n=26) and 120 minutes (r=0.46, p=0.02, n=23) confirming the glucagon secretagogue role of plasma alanine in physiological concentration. Branched free amino acids after 120 minutes were also correlated with postprandial insulin after 120 minutes (r=0.56, P=0.0061, n=22).

### DISCUSSION

The above shows that cod and soy proteins improve glucose tolerance and insulin sensitivity compared with casein in rats, as determined by indexes of glucose tolerance and insulin sensitivity during IVGTT, the test meal, and by direct measurement of peripheral insulin action using the hyperinsulinemic-euglycemic clamp technique.

Our observation that cod and soy protein diets produce lower fasting plasma glucose and insulin concentrations than the casein diet is consistent with previous results obtained in our laboratory [15] and with data published by Vahouny et al. [11], who showed lower serum insulin concentrations in fasted rats fed soy protein than in those fed casein. The reduction in both fasting glucose and insulin levels in cod- and soy protein-fed rats suggests improvement of insulin sensitivity. Similarly, the lower magnitude of the postprandial insulin response to cod and soy protein feeding in the present study is in good agreement with data from Hubbard and Sanchez [13] who reported lower blood insulin levels in humans fed a soy protein meal compared with those fed a casein meal. However, in the present study, the postprandial experiment did not allow us to distinguish between chronic (fasting) and acute (postprandial) effects of the different protein diets because the same diets were used for the acute test meal. However, the purpose of the test meal experiments was to examine the glucose, insulin, C-peptide, and glucagon responses to the dietary proteins under usual feeding conditions.

Our results strongly suggest that the reductions ofplasma insulin concentrations in cod- and soy protein-fed rats during the IVGTT and the test meal were to a large extent associated with an enhanced peripheral insulin sensitivity, as assessed by the hyperinsulinemic-euglycemic clamp technique. The greater whole body insulin action, 2-[³H]DG disappearance rate, and skeletal muscle 2-[³H]DG uptake in cod- and soy protein-fed rats clearly indicate that those rats displayed improved peripheral insulin sensitivity compared with casein-fed rats. These results further suggest that skeletal muscle, the main site of insulin-stimulated peripheral glucose disposal, is a key target for dietary protein action.

On the other hand, the greater glucoregulation in rats fed cod or soy protein could also be attributed to either decreased pancreatic insulin release or increased hepatic insulin extraction. On the one hand, insulin and C-peptide are secreted in equimolar amounts by the pancreas [38], but, in contrast to insulin, very little C-peptide is catabolized by the liver [39], allowing determination of pancreatic insulin secretion. In the present study, a lower C-peptide peak response was observed after the soy protein meal than after the casein meal, suggesting a decrease in insulin secretion in the former. On the other hand, simultaneous assessment of C-peptide and insulin concentrations in peripheral blood enabled us to estimate hepatic insulin removal, as calculated from the C-peptide-to-insulin molar ratio [40]. Hepatic insulin extraction was higher before (fasting state) and 30 min after the test meal in cod- and soy protein-fed rats than in casein-fed rats (Table 4). Therefore, it appears that, in the post-prandial state, both a lower insulin secretion and a higher hepatic insulin extraction may have contributed to reduce plasma insulin concentrations in rats fed cod and soy proteins. Additional features, such as glucose absorption from the gut and non-insulin-mediated glucose uptake, could also be involved in the increased glucose tolerance and insulin sensitivity observed in rats fed cod and soy proteins.

Hypertriglyceridemia often accompanies the development of insulin resistance and impaired glucose tolerance associated with high-sucrose diets [29], but their causal relationship is still unclear [2]. Insulin influences both the rate of hepatic triglyceride synthesis and subsequent very low density lipoprotein (VLDL) triglyceride secretion in the circulation and the rate of triglyceride disappearance from the blood stream through its action on lipoprotein lipase (LPL) activity [41]. In the present study, feeding cod and soy proteins resulted in lowered fasting and postprandial circulating triglyceride concentrations. These results are in good agreement with those published in a study from our laboratory [15] and others [11, 42] showing the hypolipidemic effect of soy protein compared with casein in rats. According to Beynen and Sugano [43], increased insulin sensitivity, such as observed in our rats fed cod and soy proteins, may decrease tissue fatty acid mobilization and, in turn, decrease synthesis and secretion of VLDL triglycerides from the liver, reducing plasma triglyceridemia. Interestingly, Demonty et al. [44] recently demonstrated that LPL activity was lower in skeletal muscle of rats fed cod and soy proteins than of those fed casein. This is expected to decrease fatty acid availability at the muscle cells and thus reduce the ratio of fat to glucose oxidation in these cells [45]. It is therefore possible that feeding cod or soy protein can reduce the supply of lipids and improve muscle insulin sensitivity compared with casein. However, whether the reduction ofplasma triglycerides was the cause or the result of improved insulin action in cod- and soy protein-fed rats remains to be investigated.

Alterations in glucagon levels could contribute to the diet-induced changes in insulin sensitivity and triglyceridemia. Indeed, postprandial glucagon concentrations were higher in the casein group than in cod protein- and soy protein-fed animals, suggesting that the daily glucagon concentrations, which are more often in the postprandial state, could induce higher hepatic glucose output and higher fasting glucose levels [46]. In addition, according to Hubbard and Sanchez [13], a high insulin-to-glucagon ratio can be considered as an early indicator of glucose intolerance. The present results corroborate this notion, since the insulin-to-glucagon ratio was higher in rats fed casein than in those fed cod or soy protein before and 30 min after the test meal.

The mechanisms by which cod and soy proteins improve glucose tolerance and insulin sensitivity are still unclear. Differences in the amino acid composition of dietary proteins have been proposed to mediate protein-dependent changes in glucose and insulin dynamics [47, 48]. An early report [18] suggested that essential amino acids, either individually or in combination, can stimulate the pancreatic release of insulin. In that report [18], arginine given alone was the most potent stimulus for the release of insulin. However, at concentrations found in dietary proteins, arginine has been rather associated with a decrease of fasting insulin levels [48]. This is in contrast with supraphysiological doses of arginine, which have been associated with increases in both insulin and glucagon concentrations [18, 49]. Mulloy et al. [50] demonstrated that a diet containing 1.0% arginine, which is in close agreement with what is found in the cod protein (1.3%) and soy protein (1.5%) diets, induces lower plasma insulin concentrations 30 and 45 min after an IVGTT than a diet containing 0.5% arginine, which is closely equivalent to the arginine level found in our casein diet (0.7%; see Ref. [16]). Furthermore, Vahouny et al. [11] showed that the addition of arginine to a casein diet, to mimic the lysine-to-arginine ratio of soy protein, resulted in serum fasting insulin levels similar to those measured in rats given a soy protein diet. More recent reports [19, 22] further suggest that amino acids can diminish insulin's ability to stimulate peripheral glucose transport. Indeed, infusion of branched-chain amino acids (leucine, isoleucine, valine), which are predominantly metabolized in skeletal muscles, has been shown to inhibit insulin-mediated glucose uptake in the forearm muscle [20]. Interestingly, the present study shows that the amount of branched-chain amino acids is slightly higher in casein than in cod and soy proteins. However, the mechanism by which dietary proteins induce insulin resistance at the cellular level is not yet well understood. Patti et al. [22] have proposed that a mixture of 20 amino acids can inhibit critical early steps in postreceptor insulin action for glucose transport, including decreased insulin-stimulated tyrosine phosphorylation of insulin receptor substrate (IRS)-1 and IRS-2, reduced binding of the p85 subunit of phosphatidylinositol 3-kinase to IRS-1 and IRS-2, and inhibition of insulin-stimulated phosphatidylinositol 3-kinase activity. It is therefore possible that specific amino acids of dietary proteins regulate skeletal muscle insulin sensitivity for glucose disposal by directly modulating the insulin signaling pathway.

In summary, the results of the present study show that, when compared with casein, soy protein and cod protein improve fasting and postprandial plasma insulin responses in rats. The beneficial effects of these proteins on glucose and insulin dynamics appear to be largely explained by an improved insulin sensitivity, as shown by an increased insulin action in skeletal muscle.

### EXAMPLE 2

### Protection from Obesity-Induced Insulin Resistance by Dietary Cod Protein

### MATERIALS AND METHODS

Materials. 2-deoxy-D-[1,2-³H]glucose (2-[³H]DG), and D-¹⁴C-sucrose was obtained from NEN Life Science Products (Boston, Mass.). Purified human insulin (Humulin R) was purchased from Eli Lilly (Indianapolis, Ind.). All diets components were from ICN Biochemicals, Cleveland, Ohio, except vitamin mix which was from Harlan Teklad, Madison, Wis. and cod protein which was prepared in our laboratory as previously described [44].

Treatment of animals. Male Wistar rats (Charles River, Montreal, Qc, Canada) weighing 200-250 g at the beginning of the study were placed on a high-fat diet for 4 wks and food was available ad libitum. The high-fat diet consisted of 20% (wt/wt) protein (either casein, soy protein or cod protein (14.7% of calories), 19.8% lard (32.7% of calories), 19.8% corn oil (32.7% of calories), 24.5% sucrose (19.9% of calories) and 5% cellulose. The high-fat diets were supplemented with 1.4% vitamin mixture, 6.7% AIN-76 mineral mix, 0.2% choline bitartrate and 0.004% BHT. Diets were identical except for the amino acid composition of the dietary protein component which differed as detailed previously [16]. The residual n-3 fatty acid content of cod protein have been measured, and while the amount of 18:3 and 22:5 were not detectable, the amounts of 20:5 and 22:6 were 114 μg/100 g and 164 μg/100 g of diet respectively. These very low amounts are considered negligible in this experiment. The energy content of the diet was measured in an automatic adiabatic calorimeter (Model 1241; Parr Instruments, Moline, Ill.) and found to be isoenergetic: casein (25.52 kJ/g), soy (25.25 kJ/g), cod (25.38 kJ/g). The protein content (N×6.25) was assayed by Kjeldahl Foss autoanalyzer (Model 1612; Foss Co., Hillerod, Denmark). The level of protein in the purified diets was adjusted to an isonitrogenous basis at the expense of carbohydrates. A control chow-fed group was studied identically to those on the high-fat diets. This group was included in this study to assess the extent of insulin resistance induce by high-fat feeding. According to the manufacturer the chow diet contained, in percent of calories, 57.3% carbohydrate, 18.1% protein and 4.5% fat and 14.3 kJ/g (Charles River rodent chow 5075, Purina Mills, Strathroy, ON, Canada). Food intake was estimated every day and body weight was measured weekly.

Whole-body Glucose Disposal and Individual Tissues Glucose Uptake Rates.

Whole-body insulin-mediated glucose disposal was assessed by the hyperinsulinemic euglycemic clamp technique, as previously described [33]. Briefly, catheters were inserted into the left jugular vein and into the right carotid artery. Rats were allowed to recover from surgery for 4-5 days before the clamp procedure was performed. Rats were fasted overnight (12-14 hours) before receiving infusion of saline (basal) or insulin at 4 mU*kg⁻¹*min⁻¹ for 140 min using a syringue pump (Razel, Stamford, Conn.). The venous catheter was used for the multiple infusions, while blood samples were obtained from the carotid artery.

In vivo basal and insulin-stimulated glucose uptake rates in individual tissues of clamped rats were determined by measuring the incorporation of 2-[³H]DG, as described in detail previously [33]. Briefly, 2-[³H]DG and ¹⁴C-sucrose were administrated together as an intravenous bolus 120 min after the start of the insulin infusion. Blood samples were obtained at 5, 7.5, 10, 12.5, 15, 17.5 and 20 min after bolus administration for determination of plasma disappearance rates of 2-[³H]DG and ¹⁴C-sucrose. At the completion of the clamp, rats were quickly killed by decapitation and tissues were rapidly removed, weight and frozen into liquid nitrogen and stored at −80° C. for subsequent analysis. The accumulation of 2-[³H]DG in tissues, corrected for extracellular space with ¹⁴C sucrose, was used as an index of glucose uptakes rates as described previously [33].

Cell culture and in vitro glucose uptake. L6 skeletal muscle cells (kind gift of Dr. A. Klip, Hospital for Sick Children,Toronto) were grown and differentiated as described previously [51]. Fully differentiated myotubes were serum-deprived 5 h prior to glucose transport experiments. Cells were incubated for 1 h in Earle's balanced salt solution (EBSS) containing mixtures of amino acids corresponding to those found in rats that consumed either casein, soy protein, cod protein or a standard chow diet. Insulin or medium alone (controls) was added during the last 45 min of the treatment. The concentration of each amino acid was previously determined in rats fed one of the three purified diets varying in protein source, namely casein, cod protein, soy protein for 28 d. Fasted rats received a 5 g meal of their assigned experimental diet for 30 min. The mean concentrations of amino acids 30 min post-meal were as follows: for casein, cod protein, soy protein and chow groups respectively: alanine, 663, 563, 501, 603 μM; arginine, 150, 194, 176, 136 μM; asparagine, 129, 111, 121, 107 μM; aspartate, 26, 21, 20, 19 μM; cysteine, 23, 29, 24, 15 μM; glutamate, 110, 114, 210, 103 μM; glutamine, 1263, 925, 1027, 1370 μM; glycine, 215, 255, 272, 381 μM; histidine, 76, 67,68, 58 μM; isoleucine, 115, 90, 98, 89 μM; leucine, 170, 122, 130, 131 μM; lysine, 470, 440, 386, 373 μM; methionine, 98, 102, 67, 76 μM; phenylalanine, 104, 76, 82, 76 μM; proline, 366, 189, 222, 284 μM; serine, 291, 217, 270, 283 μM; threonine,366, 260, 277, 386 μM; tyrosine, 112, 66, 85, 90 μM; valine, 230, 163, 166, 176 μM. After treatment with amino acids with or without insulin, cells were rinsed once with glucose-free Hepes-buffered saline solution pH 7.4 (140 mM NaCl, 20 mM Hepes-Na, 5 mM KCI, 2.5 mM MgSO4, 1 mM CaCl2) and subsequently incubated for 8 min with 10 μM 2-deoxy-D-glucose containing 0.3 μCi/ml 2-[³H]DG in the same buffer, as described previously [51].

TNF-α Protein Expression in Adipose Tissue and Skeletal Muscle.

Enzyme-linked immunosorbent assays (ELISA) were used for the detection of TNF-α in tissue extracts. Epididymal white adipose tissue was homogenized with a glass tissue grinder (Kontes, Vineland, N.J.) in lysis buffer (20 mM imidazole, pH 6.8, 100 mM KCI, 1 mM EGTA, 10 mM NaF, 0.2% Triton X-100, 1 mM PMSF and protease inhibitor cocktail) and centrifuged at 2500×g for 10 min. Mixed gastrocnemius muscle was homogenized with a polytron in 6 volumes of lysis buffer (20 mM Tris, pH 7.5, 140 mM NaCl, 1 mM MgCl2, 1 mM CaCl2, 10% glycerol, 1 mM PMSF and protease inhibitor cocktail), solubilized with 1% NP-40 for 1 hour at 4° C. and centrifuged at 14 000×g for 10 min. ELISA was carried out using a TNF-α antibody (Pharmingen Canada, Mississauga, Ont., Canada). Streptavidin-HRP conjugate and 3,3′,5,5′-tetramethylbenzidine (TMBA) were purchased from RDI (Flanders, N.J.). Secondary antibodies were biotinylated with NHS-LC-biotin (Pierce, Rockford, Ill.). Recombinant TNF-α was used for the standard curves, using an antibody against recombinant TNF-α (R&D Systems, Minneapolis, Minn.). Values for skeletal muscle were corrected for protein content determined by the bicinchoninic acid method using BSA as the standard. Adipose tissue TNF-α levels were corrected for DNA content obtained as follows. Adipose tissue was homogenized in Tris buffer (150 mM NaCl, 0.1% Triton X-100, 10 mM Tris, pH 8.0) and incubated at 37° C. for 2 hrs with 0.1% SDS 100 μg/ml proteinase K and 10 mM EDTA. DNA was extracted with 1 vol. of phenol-CHCl3 and precipitated in 2 vol. of ethanol and 0.1 vol. of 5 mM NaCl. Pelleted DNA was washed in 70% ethanol and resuspended in water. DNA content was determined spectrophotometrically (260 nM).

Analytical deternination. Plasma glucose determination was measured using the glucose oxidase method, with a Beckman glucose analyser (Beckman Instruments, Palo Alto, Calif.). Plasma insulin and leptin concentrations were measured by radioimmunoassay using a rat insulin and rat leptin specific kit from Linco (St. Louis, Mo.). Non-esterified fatty acids were determined enzymatically (Wako Chemicals, Richmond, Va.). Amino acid concentrations of plasma samples were determined after deproteinization as reported by Galibois et al [36] and were analyzed by ion-exchange chromatography using a Beckman amino acid analyzer (Palo Alto, Calif) model 6300. The residual n-3 fatty acid levels in cod protein preparation was determined by capillary gas chromatography according to the method of Luddy et al [52].

Statistical analysis. Values are expressed as mean±SE. ANOVA analyses and Duncan new multiple range test were applied to determine differences between means. P<0.05 was accepted as statistically significant. The chow-fed animal group was not included in the statistical analysis because it was only used to assess the extent of insulin resistance in high-fat fed rats.

### RESULTS

Effect of Dietary Proteins on Physiological Parameters of High-Fat Fed Rats

Body weight, energy intake, fasting and clamped plasma glucose and insulin concentrations were similar among high-fat fed dietary groups (Table 6). Fasting plasma concentrations of non-esterified free fatty acids (casein: 0.55±0.06, cod: 0.64±0.07, soy: 0.61±0.05 mM) or leptin (casein: 3.25±0.25, cod: 3.78±0.36, soy: 4.25±0.30 μg/ml) were also similar in the high-fat fed animals. As expected, all basal (pre-clamp) parameters were higher in high-fat fed rats as compared to chow-fed animals, used for comparison purposes in the present study.

Effect of Dietary Proteins on Whole-Body and Individual Tissue Insulin Action in High-Fat Fed Rats

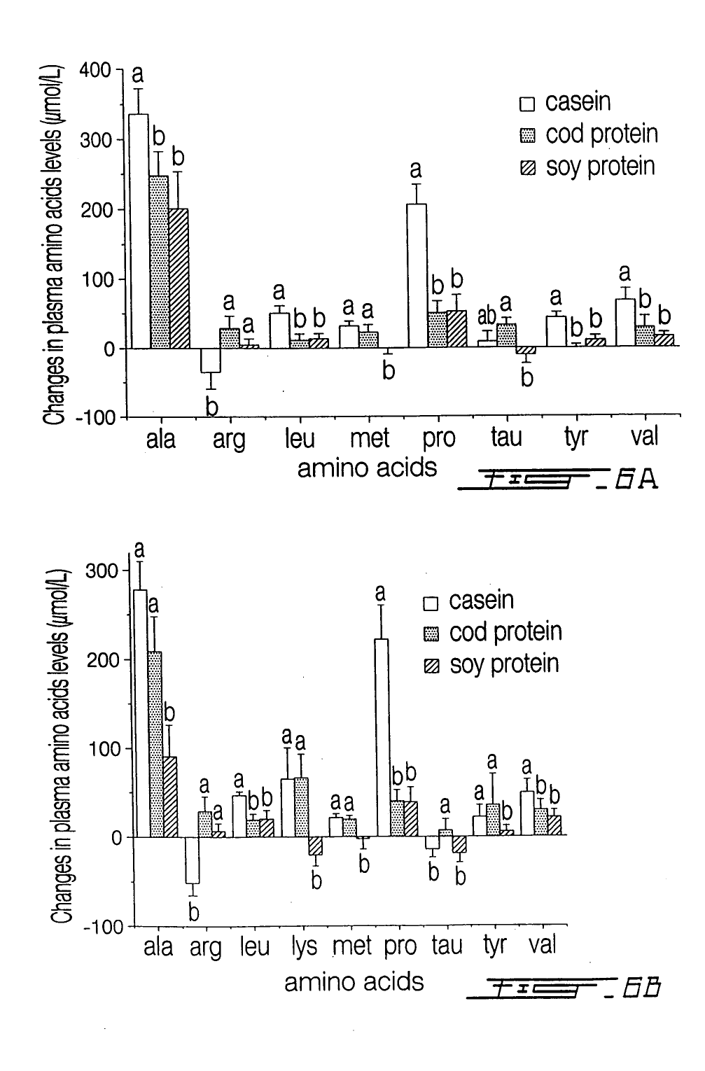

The effect of dietary proteins on insulin-mediated whole-body glucose disposal in high-fat fed rats was determined during euglycemic clamps in which plasma insulin was either kept at fasting levels (˜0.2-0.3 nM) (saline infusion) or raised to sub-physiological concentrations (˜0.7-0.8 nM) by constant infusion of insulin (4 mU*kg⁻¹*min⁻¹) (see Table 6). FIG. 7 shows the insulin-mediated glucose infusion rates (GIR₆₀₋₁₂₆) that were required to maintain pre-clamp glucose values. The GIR₆₀₋₁₂₀ was ˜40% and ˜60 % lower (P<0.05) in casein- and soy protein-fed groups, respectively, as compared to cod protein-fed obese rats. In the latter group, the GIR₆₀₋₁₂₆ was found to be similar to that observed in the reference chow-fed non-obese group (dotted line).

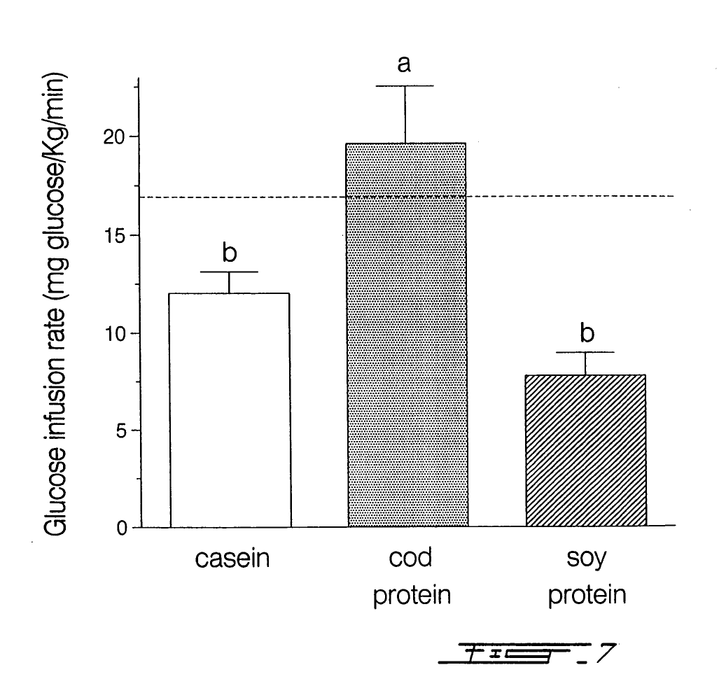

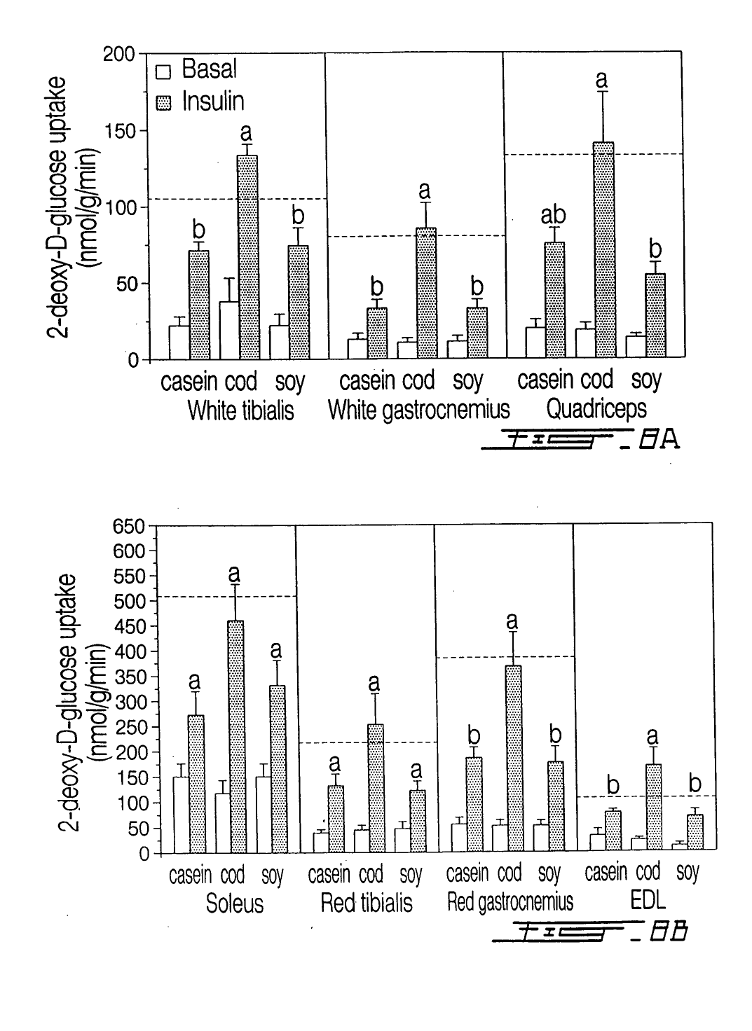

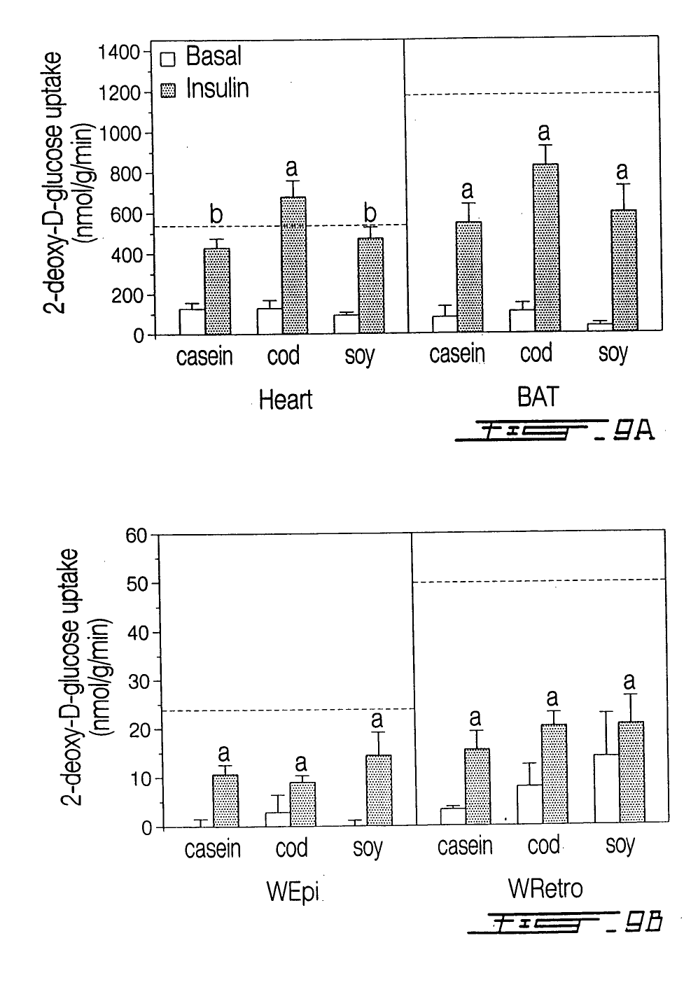

The effects of casein, cod or soy proteins on basal and insulin-stimulated glucose uptake in individual tissues of high-fat fed obese rats are shown in FIGS. 8 and 9. As compared to chow-fed lean animals (dotted line), high-fat fed obese rats consuming casein or soy proteins exhibited markedly decreased insulin-stimulated 2-[³H]DG uptake in all skeletal muscles. In stark contrast, cod protein-fed obese rats showed increased insulin action on 2-[³H]DG uptake as compared to casein or soy protein-fed groups, to a level that was comparable to that observed in chow-fed controls. The effect of cod protein on insulin-stimulated 2-[³H]DG uptake was similar in muscles enriched with either oxidative-type I fibers (e.g. soleus), oxidative-glycolytic-type Ila fibers (e.g. red gastrocnemius) or glycolytic-type IIb fibers (e.g. white gastrocnemius). Similar results were observed in cardiac muscle (FIG. 9A). Basal 2-[³H]DG uptakes in skeletal or cardiac muscles of obese rats were not affected by the source of dietary proteins (FIGS. 8-9) and were not different than that measured in chow-fed animals (data not shown).

We also examined the effects of dietary proteins on insulin-stimulated2-[³H]DG uptake in brown and white adipose tissues (FIG. 9). It should be noted that glucose uptake values were much greater in brown adipose tissue than white adipose tissues whatever the diet consumed (cf y axes in FIG. 9A vs. 9B), in accordance with previous studies [33]. High-fat feeding was associated with impaired insulin action in brown fat of both casein and soy-protein fed rats, as compared to chow-fed controls. Cod protein-fed obese animals showed an improved insulin-mediated glucose uptake, but it failed to reach the level of significance as compared to the other dietary groups. Surprisingly, cod protein feeding failed to increase insulin-stimulated glucose uptake in white adipose tissues of high-fat fed obese rats (FIG. 9B). As compared to chow-fed controls, insulin-mediated 2-[³H]DG uptake was comparably reduced in all high-fat fed groups. Basal 2-[³H]DG uptake rates in adipose tissues were not affected by dietary proteins (FIG. 9B) and were not different as compared to chow-fed rats (data not shown).

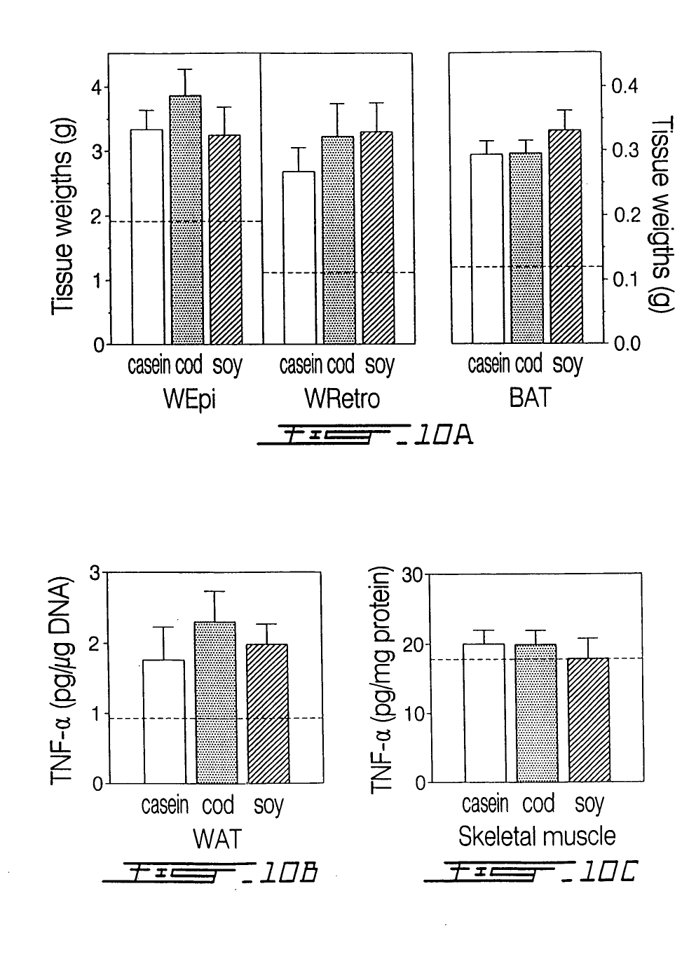

As expected, high-fat fed obese animals showed increased (2-3 times) epididymal, retroperitoneal, and interscapular brown fat weights as compared to chow-fed controls (FIG. 10A). However, no differences were observed in adiposity among dietary protein-fed groups. Moreover, the weights of the heart and skeletal muscles were not significantly different among obese rats on either protein sources and were similar to the weight of those tissues in chow-fed rats (data not shown). In accordance with previous studies [53] TNF-α protein levels were greater in adipose tissue of obese rats as compared to chow-fed rats (FIG. 10B). However, adipose tissue TNF-α levels were similar in casein, cod protein or soy protein-fed obese rats. Skeletal muscle TNF-α concentrations were also similar between obese animals on the different protein sources and were not different than that found in muscle of chow-fed rats (FIG. 10C).

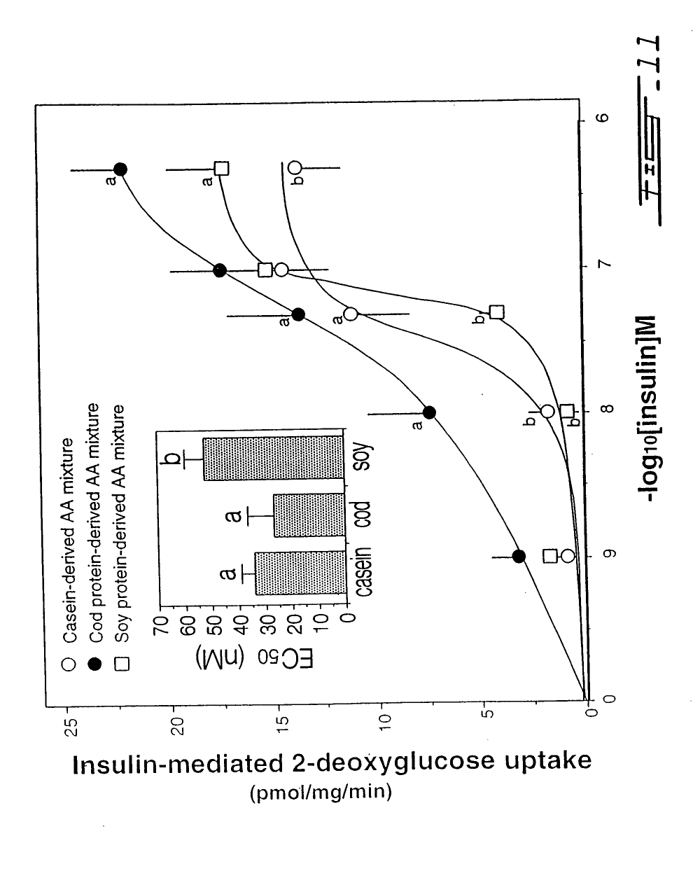

Cod protein may exert its beneficial effect on insulin sensitivity by a direct action of cod protein-derived amino acids on insulin-stimulated glucose uptake in skeletal muscle cells. To test this possibility, we have exposed cultured L6 myocytes to amino acids (AA) mixtures corresponding to the concentrations of plasma amino acids in rats fed chow, casein, cod, or soy protein diets. Cells were incubated with the AA mixtures for one hour before measuring insulin-stimulated glucose uptake rates. When compared to AA mixtures corresponding to rats fed casein or soy proteins, muscle cells exposed to the cod-derived AA mixture showed improved insulin action on glucose uptake (FIG. 11). The increasing effect of cod-derived AA was observed at all doses of insulin tested and were statistically significant at 10, 50, and 500 nM versus casein-and/or soy-derived AAs. The stimulatory effects of insulin on glucose uptake in cells exposed to cod-derived AA mixture was similar to cells incubated with AA mixture corresponding to that of chow-fed rats (data not shown). The insulin sensitivity index (EC50), calculated from individual dose-response curves, further showed that insulin sensitivity was increased in L6 myocytes exposed to the cod-derived AA as compared to the soy-derived AA mixture (insert, FIG. 11). However, no differences in insulin sensitivity were observed between muscle cells exposed to cod- or casein-derived AA mixtures.

### DISCUSSION

The results of the present study show that the consumption of dietary cod protein prevents the development of peripheral insulin resistance in rats rendered obese by feeding a high-fat/sucrose diet. Indeed, in contrast with obese rats consuming either casein or soy protein, cod protein-fed obese rats exhibited higher insulin-mediated whole-body glucose disposal rates that were comparable to that of non-obese chow-fed rats. It is well established that skeletal muscle is a major site of insulin resistance when rats are fed a high-fat/sucrose diet [54]. Measurements of individual tissue glucose uptake with 2-[³H]DG revealed that cod protein protects from the development of peripheral insulin resistance by improving insulin-stimulated glucose uptake in skeletal and cardiac muscles of high-fat fed animals. Similar results were obtained for muscles enriched with either oxidative (slow-type I), oxidative-glycolytic (fast-type (IIa) or glycolytic (fast-type IIb) fibers, suggesting that the beneficial effect of cod protein is not dependent on the metabolic or contractile nature of the muscles. Furthermore, dietary proteins failed to affect basal 2-[³H]DG uptake rates in any of the muscles investigated, thus indicating that cod protein specifically modulates insulin action on glucose uptake rather than glucose uptake per se.

It has been previously reported that n-3 fatty acids derived from fish oil improve insulin sensitivity in insulin-resistant obese rats [27, 55-58]. However, it is very unlikely that the preventive effect of cod protein on insulin resistance could be attributed to the trace amounts of n-3 fatty acids found in the purified cod protein isolate. Indeed, we have measured that the amounts of n-3 fatty acids in our cod protein diet are 150 times lower than the lowest dietary n-3 fatty acid levels that have been shown to improve insulin sensitivity in rats (6% of total calories) [27]. Moreover, our finding that the cod-derived amino acid mixture increased insulin-stimulated glucose uptake in cultured L6 myocytes indicates that at least part of the effects of cod protein on muscle insulin sensitivity is mediated by the amino acids and not the trace amounts n-3-fatty acids present in the cod protein diet.

While consuming cod protein totally abrogated the insulin-resistant effect of a high-fat/sucrose diet, feeding soy protein under the same conditions failed to prevent the occurrence of skeletal muscle insulin resistance. This is in contrast with our previous observations of a beneficial effect of soy protein on fasting insulin concentrations in rats fed a low-fat sucrose diet [59]. Moreover, we have recently observed that both cod and soy protein feeding improves glucose tolerance and insulin sensitivity in rats fed a medium-fat sucrose diet [60]. The differential effect of soy protein on insulin sensitivity in rats fed diets high or medium in fat suggests that distinct defects are responsible for the deterioration of insulin sensitivity in these insulin-resistant nutritional models and that consuming cod protein, unlike soy protein, can alleviate the metabolic defect in both models.

High-fat feeding caused marked insulin resistance for glucose uptake in both muscles and adipose tissues as compared to chow-fed animals. However, cod protein prevented the development of insulin resistance only in skeletal and cardiac muscles and not in white adipose tissues. Despite the lack of action of cod protein on white fat, insulin-mediated whole-body glucose disposal was completely normalized in obese rats consuming cod protein. These results are in line with the fact that adipose tissue contributes to a minor fraction of total glucose disposal after a meal or during insulin stimulation [61]. However, the lack of prevention of insulin resistance in adipose tissue of high-fat fed rats may help to explain the finding that fasting insulin levels are still elevated in cod protein-fed animals, despite improved insulin action in muscles. Furthermore, fasting hyperinsulinemia may also be explained by reduced hepatic insulin action or by altered insulin secretion since these defects have also been reported in high-fat fed rats [62, 63].

The beneficial effect of dietary cod protein on skeletal muscle insulin sensitivity was observed even in the face of similar body weight gain and visceral adipose tissue accretion as compared to casein- or soy protein-fed rats. These result strongly suggest that dietary cod protein prevented the causal link between visceral obesity and the development of peripheral insulin resistance. Several factors have been postulated to be responsible for the development of insulin resistance in obesity [see [64]]. One molecule that has received considerable attention is the cytokine TNF-α. There is accumulating evidence implicating TNF-α as a candidate mediator of obesity-associated insulin resistance (see [65] for a recent review). TNF-α is expressed at high levels in the enlarged adipose tissue from virtually all rodent models of obesity, as well as in obese humans [66-68]. The cytokine has also been reported to be overexpressed in muscle cells isolated from NIDDM subjects [69]. Moreover, genetic ablation of TNF-α or TNF-α function was reported to improve insulin sensitivity in various animal models of insulin resistance, including the high-fat fed mouse [69, 70]. However, there is also data to support a protective role for TNF-α receptors (p55 and p75) in obesity-linked diabetes since high-fat fed mice deficient in both receptors exhibited higher fasting hyperinsulinemia and glucose intolerance as compared to high-fat fed wild-type mice [71]. In the present study, we confirmed that TNF-α protein expression is increased in adipose tissue of high-fat fed animals. Nevertheless, our data indicate that TNF-α expression was not reduced in cod-protein fed obese rats as compared to casein- or soy protein-fed obese animals. Moreover, we found no evidence for TNF-α overexpression in skeletal muscle of high-fat fed obese rats, whatever the dietary protein sources, as compared to chow-fed animals. Taken together, these data indicate that the preventive effects of cod protein on skeletal muscle insulin resistance is unlikely to be explained by changes in adipose or muscle TNF-α expression in high-fat fed obese rats.

Another potential mechanism of obesity-associated insulin resistance is an increased availability of free fatty acids (FFA). Although we failed to observe any differences in fasting FFA levels between high-fat fed and chow-fed rats, it is likely that local lipid availability is increased in skeletal muscle of high-fat fed animals, as previously reported [63]. Elevated local lipid availability may inhibit insulin-stimulated glucose utilization through substrate competition for oxidation (the glucose-fatty acid cycle) [63] and/or by increasing the flux of fructose-6-phosphate into the hexosamine pathway [72]. In this regard, both hyperlipidemia and glucosamine infusion (used to increase the hexosamine biosynthetic pathway) have been shown to induce leptin gene expression in skeletal muscle [73]. Increased muscle leptin expression could result in impaired insulin-mediated glucose utilization since leptin increases lipid oxidation in skeletal muscle [74, 75]. On the other hand, leptin induction in skeletal muscle of high-fat fed animals may represent a protective adaptation to limit intramuscular triglyceride accumulation as recently proposed by Unger and colleagues [76, 77]. Ongoing studies are in progress to test the effects of cod protein on muscle leptin expression and intramyocellular triglyceride accumulation and their potential role in improving insulin sensitivity in high-fat fed obese rats.

Despite the possible implications of the aforementioned mechanisms in the prevention of obesity-induced insulin resistance, our finding that cod-derived amino acids can also increase insulin-stimulated glucose uptake in cultured L6 myocytes strongly suggest that a significant part of the beneficial effect of dietary cod protein on insulin-mediated glucose disposal in obese rats can be explained by a direct action of individual or groups of amino acids on skeletal muscle. To the best of our knowledge, this is the first observation that different pools of amino acids, at concentrations found in the plasma of rats fed physiological amounts of dietary proteins, can differently modulate insulin-stimulated glucose uptake in skeletal muscle cells. These effects were observed after only one hour of treatment strongly suggesting that transcriptional mechanisms are not involved in the modulatory actions of amino acids on insulin-stimulated glucose. Rather, the effects of amino acids on glucose uptake may involve acute regulation of insulin signaling events and translocation of GLUT4 transporters. In support of this, Patti and colleagues [22] recently reported that exposure of cultured muscle and hepatic cells to a balanced mixture of amino acids down-regulated early insulin signaling steps critical for glucose transport and inhibition of gluconeogenesis.

It will be important to define the role of individual or groups of amino acids in mediating the effects of cod-derived amino acid mixture on insulin-mediated glucose uptake. Several amino acids are found in different concentrations in the plasma of rats fed casein, cod, or soy proteins (see Materials & Methods, Example 2). Of these amino acids, a potentially interesting candidate is glutamine since its concentration is selectively decreased in cod protein-fed as compared to casein or soy protein-fed animals. Interest for glutamine as a potential modulator of insulin action arises from the pioneering work of Traxinger and Marshall [19] who observed a marked desensitization of the glucose transport system in adipocytes incubated in a defined buffer containing high concentrations of both insulin and glucose, plus 15 amino acids found in Dulbecco's modified Eagle's medium (DMEM). Of the 15 amino acids, glutamine was found to be fully effective in mediating loss of insulin action on glucose transport. It has been further reported that glutamine exposure also inhibits insulin-stimulated glucose transport in skeletal muscle and that it promotes insulin resistance by routing glucose through the hexosamine pathway [19, 78-82]. However, two observations made in this study argue against a role of the hexosamine pathway in the modulation of insulin action by dietary proteins. First, the hexosamine pathway is also operative in adipocytes but we found no effect of cod protein on insulin-stimulated glucose uptake in white adipose tissues. Second, the amino acid-dependent modulation of insulin action in L6 myocytes was observed within one hour of treatment, which is not likely to cause build-up of hexosamines in cells treated with casein- or soy-derived amino acid mixtures.

In summary, the present study shows that dietary cod protein prevents the development of skeletal muscle insulin resistance in high-fat fed obese rats. The beneficial action of cod protein on insulin sensitivity occurred without reductions in body weight or adiposity, strongly suggesting that cod protein protects from obesity-induced insulin resistance. The effect of dietary cod protein appears to involve, at least in part, a direct action of cod protein-derived amino acids on insulin-stimulated glucose transport in skeletal muscle cells. Interest in the present data also arises from the fact that increased cod protein consumption can be implemented in humans within guidelines of daily recommended allowances of essentials nutrients [83, 84] and thus could represent a novel nutraceutical approach to prevent the development of insulin resistance in obesity. Since insulin resistance is a central factor in visceral obesity-associated complications such as hypertension, diabetes and cardiovascular diseases [1, 64, 85], dietary cod protein may also prevent the plenium of metabolic aberrations that accompany the obese state.

Fish and/or soy dietary proteins, or their hydrolysis peptides or amino acids may be used in the preparation of compositions for use in the treatment of hyperglycemia (or diabetes) and/or insulin resistance in human and non-human animals.

When any of the active ingredients (e.g. fish and/or soy dietary proteins, or their hydrolysis peptides or amino acids) are administered orally as a suspension, these compositions are prepared according to techniques well-known in the art of pharmaceutical formulation and may contain microcrystalline cellulose for imparting bulk, alginic acid or sodium alginate as a suspending agent, methylcellulose as a viscosity enhancer, and sweeteners/flavoring agents known in the art. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, these compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The active ingredients may also be administered in intravenous (both bolus and infusion), intraperitoneal, subcutaneous, topical with or without occlusion, or intramuscular form, all using forms well known to those of ordinary skill in the pharmaceutical arts. When administered by injection, the injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these compositions may be prepared by mixing the active ingredients with a suitable non-irritating excipient, such as cocoa butter, synthetic glyceride esters or polyethylene glycols, which are solid at ordinary temperatures, but liquidify and/or dissolve in the rectal cavity to release the active ingredients. The active ingredients of the present invention may be administered as a pharmaceutical composition, for example, with an inert diluent, or with an assimilable edible carrier, or they may be enclosed in hard or soft shell capsules, or they may be compressed into tablets, or they may be incorporated directly with the food of the diet. For oral therapeutic administration, which includes sublingual administration, these active compounds maybe incorporated with excipients and used in the form of tablets, pills, capsules, ampules, sachets, elixirs, suspensions, syrups, and the like. The active compounds can also be administered intranasally as, for example, liquid drops or spray.

The effective dosage of each of the active ingredients employed in the combination may vary depending on the particular compound employed, the mode of administration, the condition being treated and the severity of the condition being treated. Thus, the dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound thereof employed. A physician, clinician or veterinarian of ordinary skill can readily determine and prescribe the effective amount of the active compounds required to prevent, counter or arrest the progress of the condition.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

These active compounds may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Although the present invention has been described hereinabove by way of preferred embodiments, it can be modified, without departing from the spirit and nature of the subject invention as defined in the appended claims.

TABLE 1
Composition of the purified diets

| Ingredients | Casein | Soy Protein | Cod Protein |
|---|---|---|---|
| Casein | 22.78 | | |
| Soy protein | | 22.90 | |
| Cod protein | | | 21.87 |
| Sucrose | 55.52 | 55.40 | 56.43 |
| Coconut oil | 10 | 10 | 10 |
| Corn oil | 1 | 1 | 1 |
| Cholesterol | 1 | 1 | 1 |
| Cellulose | 5 | 5 | 5 |
| Vitamin mix | 1 | 1 | 1 |
| Mineral mix | 3.5 | 3.5 | 3.5 |
| Choline bitartrate | 0.2 | 0.2 | 0.2 |
| Units are g/100 g. Casein, highly purified casein (ICN Biochemicals): 88% protein, 0.07% lipid; soy protein, soyprotein isolate (ICN Biochemicals): 87% protein, 0.30% lipid; cod protein, cod protein prepared in our laboratory: 91% protein, 0.19% lipid; vitamin mix, vitamin mix (Harlan Teklad) contained (mg/kg diet) 39.7 retinyl palmitate, | | | |
| #4.4 ergocalciferol, 485 a-tocopheryl acetate, 987 ascorbic acid, 110.2 i-inositol, 3,715 choline dehydrogen citrate, 49.6 menadi-one, 110.1 p-aminobenzoic acid, 99.2 niacin, 22 riboflavin, 22 pyri-doxin HCl, 22 thiamine HCl, 66.1 calcium pantothenate, 0.44 biotin, 1.98 folic acid and 29.7 vitamin B-12; mineral mix, mineral mix AIN-76 (ICN | | | |
| #Biochemicals) contained (g/kg diet) 500 CaHPO4, 74 NaCl, 220 K3C6H5O7.H2O, 52 K2SO4, 24 MgO, 3.5 MnCO3, 6.0 ferric citrate, 1.6 ZnCO3, 0.3 CuCO3, 0.01 K1O3, 0.01 Na2SeO3.5 H2O, 0.55 CrK(SO4)2.12H2O, and 118 sucrose. | | | |

TABLE 2
Amino acid composition of protein sources

| | Casein | Cod Protein | Soy Protein |
|---|---|---|---|
| Alanine | 3.40 | 6.74 | 4.49 |
| Arginine | 3.23 | 6.29 | 7.45 |
| Aspartic acid | 7.99 | 11.14 | 11.48 |
| Glutamic acid | 20.12 | 16.75 | 18.79 |
| Glycine | 2.09 | 5.39 | 4.33 |
| Histidine | 2.83 | 2.27 | 2.94 |
| Isoleucine | 3.32 | 3.24 | 3.68 |
| Leucine | 8.75 | 8.31 | 7.93 |
| Methionine | 2.04 | 1.98 | 0.66 |
| Lysine | 7.76 | 9.41 | 6.60 |
| Phenylalanine | 5.25 | 4.22 | 5.51 |
| Proline | 10.72 | 4.42 | 5.62 |
| Serine | 6.12 | 5.55 | 6.03 |
| Threonine | 4.29 | 4.84 | 4.27 |
| Tyrosine | 5.76 | 4.31 | 3.95 |
| Valine | 4.52 | 3.86 | 3.96 |
| BCAA | 16.59 | 15.41 | 15.57 |
| EAA | 38.76 | 38.13 | 35.55 |

Data are means of 3 determinations; units are g amino acid/100 g of amino acids. BCAA, sum of branched-chain amino acids: leucine, isoleucine, and valine; EAA, sum of essential amino acids: histidine, isoleucine, leucine, methionine, lysine, phenylalanine, threonine, and valine.

TABLE 3
Physiological parameters and food intake of
rats fed the purified diets

| | | Cod | Soy |
|---|---|---|---|
| | Casein | Protein | Protein |
| Food intake, | 20.8∀0.6* | 20.0∀0.5* | 20.4∀0.8* |
| g · day ⁻1 · animal ⁻1 | | | |
| (n = 20-21) | | | |
| Weight gain, | 5.4∀0.3* | 5.3∀0.3* | 5.1∀0.3* |
| g · day ⁻1 · animal ⁻1 | | | |
| (n = 20-21) | | | |

Food intake

| | | | |
|---|---|---|---|
| last meal, | 3.8∀0.8* | 3.7∀0.4* | 3.8∀0.8* |
| g/animal (n = 7-10) | | | |
| Fasting | 6.8∀0.2* | 6.1∀0.2† | 6.1∀0.2† |
| glucose, | | | |
| mmol/l (n = 20-21) | | | |
| Fasting | 0.62∀0.1* | 0.30∀0.1† | 0.31∀0.1† |
| insulin, | | | |
| nmol/l | | | |
| (n = 20-21) | | | |
| Postclamp | 6.6∀0.3* | 6.1∀0.4* | 5.5∀0.4* |
| glucose, | | | |
| mmol/l (n = 3-4) | | | |
| Postclamp | 0.98∀0.14* | 0.74∀0.14* | 0.82∀0.15* |
| insulin, | | | |
| nmol/l (n = 3-4) | | | |

Values are means ∀SE;
n, no. of rats.
Values bearing same superscript were not significantly different (P < 0.05) according to Duncan's new multiple range test.

TABLE 4
Hepatic insulin extraction (C-peptide/insulin molar ratio)
in rats fed experimental diets for 28 days

| | | Cod | Soy |
|---|---|---|---|
| | Casein | Protein | Protein |
| Fasting | 1.42∀0.13† | 2.53∀0.44* | 2.25∀0.46* |

Postprandial

| | | | |
|---|---|---|---|
| 30 min | 0.95∀0.08† | 1.50∀0.21* | 1.59∀0.25* |
| 120 min | 0.97∀0.12* | 1.01∀0.08* | 1.14∀0.13* |

Values are means ∀SE;
n = 7-10 animals.
Values bearing same superscript were not significantly different (P < 0.05) according to Duncan's new multiple range test.

TABLE 5
Plasma amino acid concentrations in fasted rats (□mol/L)

| | Casein | | Cod protein | | Soy protein | |
|---|---|---|---|---|---|---|
| Alanine | 326.26 | 19.39^{a} | 301.52 | 15.12^{a} | 305.88 | 11.77^{a} |
| Arginine | 182.94 | 23.65^{a} | 147.05 | 14.99^{a} | 170.11 | 6.69^{a} |
| Aspartic acid | 16.59 | 2.73^{b} | 14.79 | 1.17^{b} | 21.65 | 2.28^{a} |
| Citruline | 78.57 | 12.60^{a} | 54.46 | 4.18^{b} | 54.26 | 4.89^{b} |
| Glutamine | 1397.50 | 229.83^{a} | 1060.12 | 152.02^{b} | 1129.18 | 105.40^{a} |
| Glycine | 231.29 | 15.37^{b} | 268.54 | 17.80^{b} | 342.93 | 37.70^{a} |
| Histidine | 69.51 | 5.78^{a} | 53.86 | 6.46^{b} | 74.78 | 5.47^{a} |
| Leucine | 118.59 | 9.25^{a} | 105.65 | 6.86^{a} | 115.72 | 7.04^{a} |
| Lysine | 398.28 | 23.20^{a} | 391.50 | 28.15^{a} | 383.43 | 14.08^{a} |
| Methionine | 64.71 | 7.65^{a} | 62.51 | 9.68^{a} | 68.98 | 6.89^{a} |
| Proline | 158.92 | 5.91^{a} | 134.83 | 8.64^{a} | 170.38 | 13.25^{a} |
| Serine | 225.75 | 17.21^{ab} | 206.42 | 10.61^{b} | 266.25 | 13.33^{a} |
| Taurine | 94.76 | 6.51^{b} | 125.28 | 10.51^{a} | 71.71 | 7.88^{c} |
| Tyrosine | 68.53 | 8.41^{a} | 64.55 | 4.90^{a} | 76.07 | 2.98^{a} |
| Threonine | 300.27 | 25.38^{a} | 221.79 | 16.10^{b} | 267.86 | 12.32^{ab} |
| Valine | 162.52 | 14.27^{a} | 130.66 | 8.28^{a} | 151.88 | 10.60^{a} |
| LAR¹ | 2.33 | 0.25^{a} | 2.76 | 0.20^{a} | 2.27 | 0.10^{a} |
| BAA² | 355.95 | 24.45^{a} | 302.36 | 20.43^{a} | 344.22 | 21.24^{a} |
| EAA³ | 1189.78 | 64.36^{a} | 1046.87 | 62.54^{a} | 1139.62 | 36.65^{a} |
| TAA⁴ | 4273.82 | 194.54^{a} | 4120.54 | 161.30^{a} | 4335.57 | 108.62^{a} |

Values are expressed as means SEM. Groups bearing a similar superscript are not significantly different (p < 0.05).
¹LAR: Lysine arginine ratio.
²BAA: Sum of branched-chain amino acids: leucine, isoleucine and valine.
³EAA: Sum of essential amino acids except histidine.
⁴TAA: Sum of total amino acids.

TABLE 6
Physiological parameters of experimental dietary groups.

| | High-fat | | | |
|---|---|---|---|---|
| Dietary groups | Casein | Cod protein | Soy protein | Chow |
| Final body | 380 ± 20 | 400 ± 21 | 368 ± 23 | 351 ± 20 |
| weight (g) | | | | |
| Energy intake | 373 ± 18 | 378 ± 20 | 363 ± 12 | 320 ± 12 |
| (kJ · day⁻¹) | | | | |
| Pre-clamp | | | | |
| Plasma glucose | 8.9 ± 0.2 | 8.3 ± 0.3 | 8.2 ± 0.3 | 7.2 ± 0.3 |
| (mmol · L⁻¹) | | | | |
| Plasma insulin | 0.25 ± 0.03 | 0.26 ± 0.04 | 0.26 ± 0.05 | 0.13 ± 0.02 |
| (nmol · L⁻¹) | | | | |
| Hyperins. | | | | |
| Clamp¹ | | | | |
| Plasma glucose | 8.5 ± 0.4 | 8.2 ± 0.6 | 8.0 ± 0.3 | 7.2 ± 0.4 |
| (mmol · L⁻¹) | | | | |
| Plasma insulin | 0.68 ± 0.09 | 0.79 ± 0.19 | 0.69 ± 0.13 | 0.58 ± 0.08 |
| (nmol · L⁻¹) | | | | |

¹Steady-state plasma glucose and insulin during the last 60 minutes of the hyperinsulinemic-euglycemic clamp. Values are means ± SEM, n = 7-9 per group. No statistically significantly differences were observed among high-fat fed dietary groups by ANOVA analysis. Values for the chow-fed group are shown for comparison purposes.

List of Reference
1. DeFronzo, R. A., Insulin resistance: a multifaceted syndrome responsible for NIDDM, obesity, hypertension, dyslipidaemia and atherosclerosis. Neth J Med, 1997. 50(5): p. 191-7.
2. Reaven, G., Hypertriglyceridemia in the central feature of syndrome X. Cardiovasc Risk Factors, 1996. 6: p. 29-35.
3. Chisholm, K. W. and K. O'Dea, Effect of short-term consumption of a high fat diet on glucose tolerance and insulin sensitivity in the rat. J Nutr Sci Vitaminol (Tokyo), 1987. 33(5): p.377-90.
4. Harris, R. B. and H. Kor, Insulin insensitivity is rapidly reversed in rats by reducing dietaryfatfrom 40 to 30% of energy. J Nutr, 1992. 122(9): p. 1811-22.
5. Kraegen, E. W., et al., Development of muscle insulin resistance after liver insulin resistance in high-fat-fed rats. Diabetes, 1991. 40(11): p. 1397-403.
6. Sevilla, L., et al., Chronic high-fat feeding and middle-aging reduce in an additive fashion Glut4 expression in skeletal muscle and adipose tissue. Biochem Biophys Res Commun, 1997. 235(1): p. 89-93.
7. Storlien, L. H., et al., Dietary fats and insulin action. Diabetologia, 1996. 39(6): p.621-31.
8. Braaten, J. T., et al., High beta-glucan oat bran and oat gum reduce postprandial blood glucose and insulin in subjects with and without type 2 diabetes. Diabet Med, 1994. 11(3): p.312-8.
9. Storlien, L. H., et al., Effects of sucrose vs. starch diets on in vivo insulin action, thermogenesis, and obesity in rats. Am J Clin Nutr, 1988. 47(3): p. 420-7.
10. Rossetti, L., et al., Effect ofdietary protein on in vivo insulin action and liver glycogen repletion. Am J Physiol, 1989. 257(2 Pt 1): p. E212-9.
11. Vahouny, G. V., et al., Effects ofcasein and soyprotein on hepatic and serum lipids and lipoprotein lipid distributions in the rat. Atherosclerosis, 1985. 56(2): p. 127-37.
12. Galibois, I., et al., Independent effects of protein and fibre sources on glucose and cholesterol metabolism in the rat. Nutrition Research, 1992. 12: p. 643-655.
13. Hubbard, R. W. and A. Sanchez, Dietary protein control ofserum cholesterol by insulin and glucagon. Monogr Atheroscler, 1990. 16: p. 139-47.
14. Iritani, N., et al., Dietary soybean protein increases insulin receptor gene expression in Wistar fatty rats when dietary polyunsaturated fatty acid level is low. J Nutr, 1997. 127(6): p. 1077-83.
15. Hurley, C., I. Galibois, and H. Jacques, Fasting and postprandial lipid and glucose metabolisms are modulated by dietary proteins and carbohydrates: Role of plasma insulin concentrations. J Nutr Biochem, 1995. 6: p. 540-6.
16. Jacques, H., Y. Deshaies, and L. Savoie, Relationship between dietary proteins, their in vitro digestion products, and serum cholesterol in rats. Atherosclerosis, 1986. 61(2): p. 89-98.
17. Sugano, M., et al., Effects of arginine and lysine addition to casein and soya-bean protein on serum lipids, apolipoproteins, insulin and glucagon in rats. Br J Nutr, 1982. 48(2): p. 211-21.
18. Fajans, S. S., et al., Effect ofamino acids and proteins on insulin secretion in man. Recent Prog Horm Res, 1967. 23: p. 617-62.
19. Traxinger, R. R. and S. Marshall, Role of amino acids in modulating glucose-induced desensitization ofthe glucose transport system. J Biol Chem, 1989. 264(35): p.20910-6.
20. Schwenk, W. F. and M. W. Haymond, Effects ofleucine, isoleucine, or threonine infusion on leucine metabolism in humans. Am J Physiol, 1987. 253(4 Pt 1): p. E428-34.
21. Flakoll, P. J., et al., Short-term regulation ofinsulin-mediated glucose utilization in four-day fasted human volunteers: role ofamino acid availability. Diabetologia, 1992. 35(4): p.357-66.
22. Patti, M. E., et al., Bidirectional modulation of insulin action by amino acids. J Clin Invest, 1998. 101(7): p. 1519-29.
23. Nakaya, Y., et al., Taurine improves insulin sensitivity in the Otsuka Long-Evans Tokushima Fatty rat, a model of spontaneous type 2 diabetes. Am J Clin Nutr, 2000. 71(1): p. 54-8.
24. Anuradha, C. V. and S. D. Balakrishnan, Taurine attenuates hypertension and improves insulin sensitivity in thefructose-fed rat, an animal model of insulin resistance. Can J Physiol Pharmacol, 1999. 77(10): p. 749-54.
25. Kraegen, E. W., et al., In vivo insulin resistance in individual peripheral tissues of the high fat fed rat: assessment by euglycaemic clamp plus deoxyglucose administration. Diabetologia, 1986. 29(3): p. 192-8.
26. Storlien, L. H., et al., Fat feeding causes widespread in vivo insulin resistance, decreased energy expenditure, and obesity in rats. Am J Physiol, 1986. 251(5 Pt 1): p. E576-83.
27. Storlien, L. H., et al., Influence of dietary fat composition on development of insulin resistance in rats. Relationship to muscle triglyceride and omega-3 fatty acids in muscle phospholipid. Diabetes, 1991. 40(2): p.280-9.
28. Ikemoto, S., et al., High fat diet-induced hyperglycemia: prevention by low level expression of a glucose transporter (GLUT4) minigene in transgenic mice. Proc Natl Acad Sci U S A, 1995. 92(8): p. 3096-9.
29. Boivin, A. and Y. Deshaies, Dietary rat models in which the development of hypertriglyceridemia and that of insulin resistance are dissociated. Metabolism, 1995. 44(12): p. 1540-7.
30. Han, D. H., et aL, Insulin resistance of muscle glucose transport in rats fed a high-fat diet: a reevaluation. Diabetes, 1997. 46(11): p. 1761-7.
31. Carroll, J. F., et al., Hypertension, cardiac hypertrophy, and neurohumoral activity in a new animal model of obesity. Am J Physiol, 1996. 271(1 Pt 2): p. H373-8.
32. Storlien, L. H., et al., Syndromes of insulin resistance in the rat. Inducement by diet and amelioration with benfluorex. Diabetes, 1993. 42(3): p. 457-62.
33. Roy, D., M. Perreault, and A. Marette, Insulin stimulation of glucose uptake in skeletal muscles and adipose tissues in vivo is NO dependent. Am J Physiol, 1998. 274(4 Pt 1): p. E692-9.
34. Shibata, H., et al., Cold exposure reverses inhibitory effects offasting on peripheral glucose uptake in rats. Am J Physiol, 1989. 257(1 Pt 2): p. R96-101.
35. Vallerand, A. L., F. Perusse, and L. J. Bukowiecki, Cold exposure potentiates the effect of insulin on in vivo glucose uptake. Am J Physiol, 1987. 253(2 Pt 1): p. El 79-86.
36. Galibois, I., et aL, Net appearance ofamino acids in portal blood during the digestion of casein or rapeseedproteins in thepig. Can J Physiol Pharmacol, 1989. 67(11): p.1409-17.
37. Collier, G. R., et al., The acute effect offat on insulin secretion. J Clin Endocrinol Metab, 1988. 66(2): p. 323-6.
38. Horwitz, D. L., et al., Proinsulin, insulin, and C-peptide concentrations in human portal and peripheral blood. J Clin Invest, 1975. 55(6): p. 1278-83.
39. Faber, O. K., et al., Kinetics of human C-peptide in man. Diabetes, 1978. 27(Suppl 1): p. 207-9.
40. Faber, O. K., et al., Decreased insulin removal contributes to hyperinsulinemia in obesity. J Clin Endocrinol Metab, 1981. 53(3): p. 618-21.
41. Howard, B. V. and W. J. Howard, Dyslipidemia in non-insulin-dependent diabetes mellitus [published erratum appears in Endocr Rev 1994 Aug;15(4):438]. Endocr Rev, 1994. 15(3): p. 263-74.
42. Iritani, N., et al., Soybean protein suppresses hepatic lipogenic enzyme gene expression in Wistarfatty rats. J Nutr, 1996. 126(2): p. 380-8.
43. Beynen, A. C. and M. Sugano, Dietary protein as a regulator oflipid metabolism: state of the art and newperspectives. J Nutr Sci Vitaminol (Tokyo), 1990. 36 Suppl 2: p. S1 85-8.
44. Demonty, I., Y. Deshaies, and H. Jacques, Dietary proteins modulate the effects offish oil on triglyceridemia in the rat [In Process Citation]. Lipids, 1998. 33(9): p. 913-21.
45. Randle, P.J., et al., The glucose fatty-acid cycle its role in insulin sensitivity and the metabolic disturbances of diabetes mellitus. Lancet, 1963. l(April 13): p. 785-94.
46. Felig, P., et al., Insulin, glucagon, and somatostatin in normal physiology and diabetes mellitus. Diabetes, 1976. 25(12): p. 1091-9.
47. Sanchez, A. and R. W. Hubbard, Plasma amino acids and the insulin/glucagon ratio as an explanation for the dietary protein modulation of atherosclerosis. Med Hypotheses, 1991. 35(4): p. 324-9.
48. Sugano, M., N. Ishiwaki, and K. Nakashima, Dietaryprotein-dependent modification of serum cholesterol level in rats. Significance of the arginin/lysine ratio. Ann Nutr Metab, 1984. 28(3): p. 192-9.
49. Palmer, J. P., R. M. Walter, and J. W. Ensinck, Arginine-stimulated acute phase of insulin and glucagon secretion. I. in normal man. Diabetes, 1975. 24(8): p. 735-40.
50. Mulloy, A. L., F. W. Kari, and W. J. Visek, Dietary arginine, insulin secretion, glucose tolerance and liver lipids during repletion of protein-depleted rats. Horm Metab Res, 1982. 14(9): p. 471-5.
51. Bedard, S., B. Marcotte, and A. Marette, Cytokines modulate glucose transport in skeletal muscle by inducing the expression of inducible nitric oxide synthase. Biochem J, 1997. 325(Pt 2): p. 487-93.
52. Luddy, F., et al., Rapid and quantitative procedurefor the preparation of methyl esters of butteroil and other fats. J Am Oil Chem Soc, 1968. 45: p. 549-52.
53. Spiegelman, B. M. and G. S. Hotamisligil, Through thick and thin: wasting, obesity, and TNF alpha. Cell, 1993. 73(4): p. 625-7.
54. Storlien, L. H., et al., High fat diet-induced insulin resistance. Lessons and implications from animal studies. Ann N Y Acad Sci, 1993. 683: p. 82-90.
55. Storlien, L. H., et al., Fish oil prevents insulin resistance induced by high-fat feeding in rats. Science, 1987. 237(4817): p. 885-8.
56. Mori, Y., et al., Influence of highly purified eicosapentaenoic acid ethyl ester on insulin resistance in the Otsuka Long-Evans Tokushima Fatty rat, a model of spontaneous non-insulin-dependent diabetes mellitus. Metabolism, 1997. 46(12): p. 1458-64.
57. Sohal, P. S., V. E. Baracos, and M. T. Clandinin, Dietary omega 3 fatty acid alters prostaglandin synthesis, glucose transport and protein turnover in skeletal muscle of healthy and diabetic rats. Biochem J, 1992. 286(Pt 2): p. 405-11.
58. Mori, Y., et al., Effect of highly purified eicosapentaenoic acid ethyl ester on insulin resistance and hypertension in Dahl salt-sensitive rats. Metabolism, 1999. 48(9): p. 1089-95.
59. Hurley, C., et al, Soyprotein isolate in the presence of cornstarch reduces bodyfat gain in rats. Can J Physiol Pharmacol, 1998. 76(10-11): p. 1000-7.
60. Lavigne, C., A. Marette, and H. Jacques, Cod and soy proteins compared with casein improve glucose tolerance and insulin sensitivity in rats. Am J Physiol, 2000. 278(3): p. E491-E500.
61. James, D. E., K. M. Burleigh, and E. W. Kraegen, Time dependence of insulin action in muscle and adipose tissue in the rat in vivo. An increasing response in adipose tissue with time. Diabetes, 1985. 34(10): p. 1049-54.
62. Ahren, B., et al., Islet perturbations in rats fed a high-fat diet [In Process Citation]. Pancreas, 1999. 18(1): p. 75-83.
63. Oakes, N. D., et al., Mechanisms of liver and muscle insulin resistance induced by chronic high-fat feeding. Diabetes, 1997. 46(11): p. 1768-74.
64. Despres, J. P. and A. Marette, Obesity and insulin resistance: Epidimiologic, metabolic, and molecular aspects., in Insulin resistance: The Metabolic Syndrome X, G.M.R.a.A. Law, Editor. 1999, The Humana Press. p. 51-81.
65. Hotamisligil, G. S., Mechanisms of TNF-alpha-induced insulin resistance. Exp Clin Endocrinol Diabetes, 1999. 107(2): p. 119-25.
66. Hotamisligil, G. S., et al., Increased adipose tissue expression of tumor necrosisfactor-alpha in human obesity and insulin resistance. J Clin Invest, 1995. 95(5): p. 2409-15.
67. Hotamisligil, G. S., et al., Reduced tyrosine kinase activity of the insulin receptor in obesity-diabetes. Central role of tumor necrosis factor-alpha. J Clin Invest, 1994. 94(4): p. 1543-9.
68. Kern, P. A., et al., The expression of tumor necrosis factor in human adipose tissue. Regulation by obesity, weight loss, and relationship to lipoprotein lipase. J Clin Invest, 1995. 95(5): p. 2111-9.
69. Uysal, K. T., et al., Protection from obesity-induced insulin resistance in mice lacking TNF-alphafunction. Nature, 1997. 389(6651): p. 610-4.
70. Ventre, J., et al., Targeted disruption of the tumor necrosis factor-alpha gene: metabolic consequences in obese and nonobese mice. Diabetes, 1997. 46(9): p. 1526-31.
71. Schreyer, S. A., S. C. Chua, Jr., and R. C. LeBoeuf, Obesity and diabetes in TNF-alpha receptor- deficient mice. J Clin Invest, 1998. 102(2): p.402-11.
72. Hawkins, M., et al., Role of the glucosamine pathway in fat-induced insulin resistance. J Clin Invest, 1997. 99(9): p. 2173-82.
73. Rossetti, L. and M. R. Lauglin, Correction of chronic hyperglycemia with vanadate, but not with phlorizin, normalizes in vivo glycogen repletion and in vitro glycogen synthase activity in diabetic skeletal muscle. J Clin Invest, 1989. 84(3): p. 892-9.
74. Muoio, D. M., et al., Leptin directly alters lipid partitioning in skeletal muscle [published erratum appears in Diabetes October 1997; 46(10):1663]. Diabetes, 1997. 46(8): p.1360-3.
75. Muoio, D. M., et al., Leptin opposes insulin's effects on fatty acid partitioning in muscles isolated from obese ob/ob mice. Am J Physiol, 1999. 276(5 Pt 1): p. E913-21.
76. Shimabukuro, M., et al., Direct antidiabetic effect of leptin through triglyceride depletion of tissues. Proc Natl Acad Sci U S A, 1997. 94(9): p.4637-41.
77. Unger, R. H., Y. T. Zhou, and L. Orci, Regulation of fatty acid homeostasis in cells: novel role of leptin. Proc Natl Acad Sci U S A, 1999. 96(5): p. 2327-32.
78. Marshall, S. V. Bacote, and R. R. Traxinger, Complete inhibition of glucose-induced desensitization of the glucose transport system by inhibitors of mRNA synthesis. Evidence for rapid turnover of glutaminefructose-6-phosphate amidotransferase. J Biol Chem, 1991. 266(16): p. 10155-61.
79. Traxinger, R. R. and S. Marshall, Coordinated regulation of glutamine fructose-6-phosphate amidotransferase activity by insulin, glucose, and glutamine. Role of hexosamine biosynthesis in enzyme regulation. J Biol Chem, 1991. 266(16): p.10148-54.
80. Marshall, S. V. Bacote, and R. R. Traxinger, Discovery of a metabolic pathway mediating glucose-induced desensitization of the glucose transport system. Role of hexosamine biosynthesis in the induction of insulin resistance. J Biol Chem, 1991. 266(8): p. 4706-12.
81. Hebert, L. F., Jr., et al., Overexpression of glutamine fructose-6-phosphate amidotransferase in transgenic mice leads to insulin resistance. J Clin Invest, 1996. 98(4): p. 930-6.
82. Buse, M. G., et al., Increased activity of the hexosamine synthesis pathway in muscles of insulin-resistant ob/ob mice. Am J Physiol, 1997. 272(6 Pt 1): p. E1080-8.
83. Jacques, H. L. Noreau, and S. Moorani, Effects on plasma lipoproteins and endogenous sex hormones of substituting lean whitefish for other animal-protein sources in diets of postmenopausal women. Am J Clin Nutr, 1992. 55(4): p. 896-901.
84. Gascon, A., et al., Plasma lipoprotein profile and lipolytic activities in response to the substitution of lean whitefish for other animal protein sources in premenopausal women. Am J Clin Nutr, 1996. 63(3): p. 315-21.
85. Baron, A. D., Pathogenesis and measurement of insulin resistance in hypertension. Curr Opin Nephrol Hypertens, 1994. 3(6): p. 631-5.

1. A method for potentiating normal or deficient insulin function on glucose metabolism in a mammal when measured on an intravenous glucose tolerance test, said method comprising the administration of an effective amount of one or more compounds selected from the group consisting of fish protein, hydrolyzed fish protein and fish protein amino acids.

2. A method, as defined in claim 1, wherein said one or more compounds is administered in a quantity that is about 4% to about 60% of the mammal's diet.

3. A method as defined in claim 1, wherein said deficient insulin function is caused by hyperglycemia.

4. A method as defined in claim 1, wherein said fish protein is cod protein.

5. A method, as defined in claim 1, wherein said amino acids comprise the following weight proportion: about 6.74 alanine per 100 units of total amino acids; about 6.29 arginine per 100 units of total amino acids; about 11.14 aspartic acid per 100 units of total amino acids; about 16.75 glutamic acid per 100 units of total amino acids; about 5.39 glycine per 100 units of total amino acids; about 2.27 histidine per 100 units of total amino acids; about 3.24 isoleucine per 100 units of total amino acids; about 8.31 leucine per 100 units of total amino acids; about 1.98 methionine per 100 units of total amino acids; about 9.41 lysine per 100 units of total amino acids; about 4.22 phenylalanine per 100 units of total amino acids; about 4.42 proline per 100 units of total amino acids; about 5.55 serine per 100 units of total amino acids; about 4.84 threonine per 100 units of total amino acids; about 4.31 tyrosine per 100 units of total amino acids; and about 3.86 valine per 100 units of total amino acids.

6. A method as defined in claim 5, wherein said deficient insulin function is caused by hyperglycemia.

7. A method as defined in claim 6, wherein said compounds are combined with a pharmaceutically-acceptable carrier, adjuvant or vehicle.

8. A method as defined in claim 1, wherein said hyperglycemia is the result of Type 1 or Type 2 diabetes.

9. A method as defined in claim 6, wherein said hyperglycemia is the result of Type 1 or Type 2 diabetes.

10. A method as defined in claim 3, wherein said fish protein is cod protein.

11. A method as defined in claim 11, wherein said fish protein is cod protein.

12. A method as defined in claim 9, wherein said compounds are combined with a pharmaceutically-acceptable carrier, adjuvant or vehicle.

13. A method as defined in claim 10, wherein said compounds are combined with a pharmaceutically-acceptable carrier, adjuvant or vehicle.

14. A method as defined in claim 11, wherein said compounds are combined with a pharmaceutically-acceptable carrier, adjuvant or vehicle.

15. A method, as defined in claim 1, wherein said deficient insulin function is caused by obesity.
